SEQUENCE LISTING (32 sequences)

SEQ ID NO: 1 - DNA, 909 bp, Trichoderma reesei
atgccttcagtcaaagaaacacttactcttttgctgagccaagctttccttgccactggcagcccagtagatggagagaccgttgtgaagcgacaatgcccggctattcacgtcttcggcgcccgtgaaacgacagtgagtcagggatatggctcgtccgctaccgttgtaaacttggtcatccaagcccatcccggaaccacatccgaggcaattgtctacccggcgtgcggtggtcaggcttcatgtggcggcatcagctatgctaactcagttgtgaacggaaccaacgccgcggcggcggcaatcaacaactttcacaactcttgcccggacactcagctggtgttggtcggatactcgcagggtgctcaaatcttcgataatgccctctgcggaggaggcgatcctggtgaaggaattaccaacactgctgtccccctaactgcgggagcagtttccgctgttaaagcagcaatcttcatgggagaccctcgaaacattcatggcctgccctataacgtcggaacctgtactacccagggtttcgacgcccgtccggctggcttcgtctgtcccagcgcgtccaagatcaagtcttactgcgatgccgcagacccgtactgctgcaccggaaatgaccccaacgttcaccaaggttacggccaggagtacggccagcaggctctcgctttcatcaacagccagctctcctcaggcggttctcaacccccgggcggcggcccgaccagcacctccaggccaaccagcacaagaactggaagctctcctggcccaactcagactcattggggtcagtgcggaggccagggctggaccggtcctacgcaatgtgagagcggcacgacttgccaggttatcagccaatggtactctcagtgcttgtaa

SEQ ID NO: 2 - protein, 302 aa, Trichoderma reesei
MPSVKETLTLLLSQAFLATGSPVDGETVVKRQCPAIHVFGARETTVSQGYGSSATVVNLVIQAHPGTTSEAIVYPACGGQASCGGISYANSVVNGTNAAAAAINNFHNSCPDTQLVLVGYSQGAQIFDNALCGGGDPGEGITNTAVPLTAGAVSAVKAAIFMGDPRNIHGLPYNVGTCTTQGFDARPAGFVCPSASKIKSYCDAADPYCCTGNDPNVHQGYGQEYGQQALAFINSQLSSGGSQPPGGGPTSTSRPTSTRTGSSPGPTQTHWGQCGGQGWTGPTQCESGTTCQVISQWYSQCL

SEQ ID NO: 3 - DNA, 1050 bp, Volvariella volvacea
atgattctgaggatcctcaacctcgttatgttggccatcctggccaggaccacctttgctcagcttcagcaagttaccaactttggtcccaaccctaccaatgtcgggatatacgcataccgtccatcaggcctcccagcgaaccctgcactgattgtcgccatgcactactgcactggtaccgctcaagcctactactccggaacgcgatatgcgactctcgccaacacctacaaattcatcgtcatctaccccaatgcacctgattccggaggctgttgggatgtccacaccaacgctaccctcacccacgatgcgggaggtgactcgttgggtattgcttcggcaattaggtacgcgatcaacacctggggtgtcgaccgcaaccgcgttttcgccactggtacatcgtcgggcgccatgatgaccaacgtcctggccggtgcataccccgacttgattaaagctggtgcggcgtttgctggagttccctacggttgcttcgctggccctggcatgtggaatagccaatgcgctcaaggtcaattgaccaagactgctcaacaatggggtgaccaagtccgttcgggctatcccggctatactggcagccgccccaagatgcaaatctggcatggaactcgtgacgagaccctcaactacaataacttcgacgaagcgatcaagcaatggactaacatcttcgggtacagcacgaccgcaacacaagtccaacaaaactcccctctctcaggctggacccgcagcacttatggccccaacttccaagccattagcgctgccaatgttccacacaacattcccgtccaggagaacgacgttttgaactggttcggtatcactggtggaggcccaaccaccaccactaccaccactggccctactcagcctcccacaacgacaacacctggcggtcctctccagacacaatggggccagtgcggaggaattggctggactggaccaactgcttgccaacctcccttcacttgccaatacactaacgactggtactctcagtgccgttaa

SEQ ID NO: 4 - protein, 349 aa, Volvariella volvacea
MILRILNLVMLAILARTTFAQLQQVTNFGPNPTNVGIYAYRPSGLPANPALIVAMHYCTGTAQAYYSGTRYATLANTYKFIVIYPNAPDSGGCWDVHTNATLTHDAGGDSLGIASAIRYAINTWGVDRNRVFATGTSSGAMMTNVLAGAYPDLIKAGAAFAGVPYGCFAGPGMWNSQCAQGQLTKTAQQWGDQVRSGYPGYTGSRPKMQIWHGTRDETLNYNNFDEAIKQWTNIFGYSTTATQVQQNSPLSGWTRSTYGPNFQAISAANVPHNIPVQENDVLNWFGITGGGPTTTTTTTGPTQPPTTTTPGGPLQTQWGQCGGIGWTGPTACQPPFTCQYTNDWYSQCR

SEQ ID NO: 5 - DNA, 975 bp, Thermotoga maritima
atggccttcttcgatttaccactcgaagaactgaagaaatatcgtccagagcggtacgaagagaaagacttcgatgagttctgggaagagacactcgcagagagcgaaaagttccccttagaccccgtcttcgagaggatggagtctcacctcaaaacagtcgaagcgtacgatgtcaccttctccggatacaggggacagaggatcaaagggtggctccttgttccaaaactggaagaagaaaaacttccctgcgttgtgcagtacataggatacaacggtggaagaggattccctcacgactggctgttctggccttctatgggttacatatgtttcgtcatggatactcgaggtcagggaagcggctggctgaaaggagacacaccggattaccctgagggtcccgttgaccctcagtatccaggattcatgacaagaggaatactggatcccagaacttactactacagacgagtcttcacggacgctgtcagagccgttgaagctgctgcttcttttcctcaggtagatcaagaaagaatcgtgatagctggaggcagtcagggtggcggaatagcccttgcggtgagcgctctctcaaagaaagcaaaggctcttctgtgcgatgtgccgtttctgtgtcacttcagaagagcagtacagcttgtggatacgcatccatacgcggagatcacgaactttctaaagacccacagagacaaggaagaaatcgtgttcaggactctttcctatttcgatggagtgaacttcgcagccagagcgaagatccctgcgctgttttctgtgggtctcatggacaacatttgtcctccttcaacggttttcgctgcctacaattactacgctggaccgaaggaaatcagaatctatccgtacaacaaccacgagggaggaggctctttccaagcggttgaacaggtgaaattcttgaaaaaactatttgagaaaggc

SEQ ID NO: 6 - protein, 325 aa, Thermotoga maritima
MAFFDLPLEELKKYRPERYEEKDFDEFWEETLAESEKFPLDPVFERMESHLKTVEAYDVTFSGYRGQRIKGWLLVPKLEEEKLPCVVQYIGYNGGRGFPHDWLFWPSMGYICFVMDTRGQGSGWLKGDTPDYPEGPVDPQYPGFMTRGILDPRTYYYRRVFTDAVRAVEAAASFPQVDQERIVIAGGSQGGGIALAVSALSKKAKALLCDVPFLCHFRRAVQLVDTHPYAEITNFLKTHRDKEEIVFRTLSYFDGVNFAARAKIPALFSVGLMDNICPPSTVFAAYNYYAGPKEIRIYPYNNHEGGGSFQAVEQVKFLKKLFEKG

SEQ ID NO: 7 - DNA, 1221 bp, Phanerochete Crysosporium
atggctttttcttggttgtcttttgttttgttggctttgcctgttttggctttggctagaccttctgaacatgaagctagatctttgttttgttctactccttctaatattccttttaatgatgataagttgcctgatccttttaagtttaatgatggatctcctgttagatcttttgctgattgggattgtagaagacaacaattgtctgctttgattcaaggatatgaagctggaactttgcctcctagacctcctgttgttacttctacttttactaagtctggaactactggaaatttgactgttactgctggatttcctggaaagactattactttttcttctcctattacttttcctactggaactgctccttttggaggatggcctttggttattgcttatggaggagtttctattcctattcctgatggaattgctgttttgacttatgataattctgctatggctgaacaaaatgatcaatcttctagaggagttggattgttttttgatgtttatggagctaatgctactgcttcttctatgactgcttgggtttggggattgtctagaattattgattctttggaagttactcctgctgctcatattaatactgctaagattgctgttactggatgttctagaaatggaaagggagctttgatggctggagcttttgaagaaagaattgctttgactattcctcaagaatctggatctggaggagatacttgttggagattgtctaagtttgaacaagattctggagatgttgttcaacaagctactgaaattgttcaagaaaatgtttggttttctactaattttgataattatgtttttaatatttctttgttgccttatgatcatcatgaattggctgctttggttgctcctagacctttgatttcttatgaaaatactgattttgaatggttgtctcctttgtctggatttggatgtatgactgctgctcatactgtttgggaagctatgggaattcctgataagcatggatttgttcaagttggaaatcattctcattgtgattttccttcttctttgaatcctactttgtttgctttttttgataagtttttgttgggaaaggaagctaatacttctatttttgaaactaatggattgtttaatggaactgaatgggttgcttctcaatggattaattggactactcctagatttactttgttg

SEQ ID NO: 8 - protein, 407 aa, Phanerochete Crysosporium
MAFSWLSFVLLALPVLALARPSEHEARSLFCSTPSNIPFNDDKLPDPFKFNDGSPVRSFADWDCRRQQLSALIQGYEAGTLPPRPPVVTSTFTKSGTTGNLTVTAGFPGKTITFSSPITFPTGTAPFGGWPLVIAYGGVSIPIPDGIAVLTYDNSAMAEQNDQSSRGVGLFFDVYGANATASSMTAWVWGLSRIIDSLEVTPAAHINTAKIAVTGCSRNGKGALMAGAFEERIALTIPQESGSGGDTCWRLSKFEQDSGDVVQQATEIVQENVWFSTNFDNYVFNISLLPYDHHELAALVAPRPLISYENTDFEWLSPLSGFGCMTAAHTVWEAMGIPDKHGFVQVGNHSHCDFPSSLNPTLFAFFDKFLLGKEANTSIFETNGLFNGTEWVASQWINWTTPRFTLL

SEQ ID NO: 9 - DNA, 1416 bp, Phanerochete Crysosporium
atgaagtctgctgcttatttggctgctttggctgctgttttgcctgcttatgttaatgctcaagctcaagaatggggacaatgtggaggaattggatggactggagctactacttgtgtttctggaactgtttgtactgttttgaatccttattattctcaatgtttgcctggaactgctactactgctcctcctcctcctcctcctcctcctacttctgtttcttcttcttcttcttcttctacttcttctgctcctccttctggaccttctggaacttctcctacttgttctgttgcttctactattcctggattttctaatgctgctttgcctaatccttttgtttttaatgatggatctcctgttcaatctaaggctgattttacttgtagacaacaacaaattttggctttgattcaaggatatgaagctggagctttgcctggacctcctcaatctgttactgcttctttttctaagtctggatctactggaactttgtctattactgttactgataatggaaagtctatttcttttgctcctactatttctattccttctggaactcctcctgctaatggatggcctttggttattgcttttgaaggaggatctattcctattcctgctggaattgctaagttgacttattctaattctgatatggctcaacaaactgatacttcttctagaggaaagggattgttttataatttgtatggatctggagctactgcttctgctatgactgcttgggcttggggagtttctagaattattgatgctttggaaaagactccttctgctcaaattaatactcaaagaattgctgttactggatgttctagagatggaaagggagctttgatggctggagctttggaacctagaattgctttgactattcctcaagaatctggatctggaggagatacttgttggagattgtctaaggctgaatctgatcaaggacatcaagttcaaactgctactgaaattgttactgaaaatgtttggttttctactaattttaataattatgttaataatttgaatgttttgccttatgatcatcatatgttgatggctttggttgctcctagagctttggtttcttttgaaaatactgattatacttggttgtctcctatgtctgcttggggatgtgttaatgctgctcatactgttttttctgctttgggagttgctgatcatcatggatttgctcaagttggaggacatgctcattgtgcttggcctgattctttgactccttctttgaatgctttttttaatagatttttgttggatcaaaatgttgatactaatgtttttactactaataatcaatttggaggagctacttggactcaatcttcttggattaattggtctactcctactttgtct

SEQ ID NO: 10 - protein, 472 aa, Phanerochete Crysosporium
MKSAAYLAALAAVLPAYVNAQAQEWGQCGGIGWTGATTCVSGTVCTVLNPYYSQCLPGTATTAPPPPPPPPTSVSSSSSSSTSSAPPSGPSGTSPTCSVASTIPGFSNAALPNPFVFNDGSPVQSKADFTCRQQQILALIQGYEAGALPGPPQSVTASFSKSGSTGTLSITVTDNGKSISFAPTISIPSGTPPANGWPLVIAFEGGSIPIPAGIAKLTYSNSDMAQQTDTSSRGKGLFYNLYGSGATASAMTAWAWGVSRIIDALEKTPSAQINTQRIAVTGCSRDGKGALMAGALEPRIALTIPQESGSGGDTCWRLSKAESDQGHQVQTATEIVTENVWFSTNFNNYVNNLNVLPYDHHMLMALVAPRALVSFENTDYTWLSPMSAWGCVNAAHTVFSALGVADHHGFAQVGGHAHCAWPDSLTPSLNAFFNRFLLDQNVDTNVFTTNNQFGGATWTQSSWINWSTPTLS

SEQ ID NO: 11 - DNA, 1425 bp, Chaetomium globosum
atgttgtctcctgccctcgcctcgtcccttctggtcgtactcggcgccgccggtgctcacgcccaacagcgccaagccctctggggccaatgcggtggttcaggctggaatggcccaacagcgtgtgtggatggggcatactgcttccaacagaaccaatggtaccaccagtgtatcccggggagcgaccgttcaccgagcccaacaaatccgcctcagcagtcctccacgctcaccacgtcccgggtgactgttgcacctcctacaagcacgtcgcctgcgccgggcggaggtggtactacctgccccgccactccgagcggccaaggcagcccagttgccaacgcgctcaacgaccccttcaccttccacggtggcggcaaggtatccagcaaagcagactggacgtgtcgccaagccgagatcagcaagcttctccagcaatacgagctgggcactctcccgcccaagccgtcgtcggtcacagccagcttctcgggcagcactctgagcatcagtgtctcggagggcggcaagtccatctccttctcggtgagcatcaacaaccggccgtcgagtagcggacctcacccggccatcatcaactttggaacctttggtggtcttcccgtcccggccggcgtggccaccatcaacttcaacaacgacgagattgcccagcaacaggaccagtcttcgcgcggcaagggcaagttctatgacatctacggcagcggccactctgccggcgcgctgacggcctgggcatggggtgtttcccgtatcatcgacgcgctggaactcacgcgtgctcagaccaacattgatccggcaagaatcggcgtgacaggctgctcgaggaacggcaagggcgccatggttgcgggtgcactggagcctcgcattgccctgactcttcctgaggaatctggcgcgggcggttccggctgctggagaattgctacatggcagaagaacaggggcgagaacgtgcaggagtccaagcagatcgtcacggagaacgtctggttttcgaccaacttcaacagccaggtcaacaacgtcaacgcgctgcccttcgaccaccacatgttagccggtctcatcgcgccccgtgccctttacgtcatggagaacaccgacatggagtggctcggcaaggtgtcgacctacggctgcatgggcattgcccgcaagcagtgggaggccctgggcgccctcgacagcttcggttactcgcaggttggcggtaactcccactgcagcttccccagctcgcagcagggcagcgagctcaacgcctttattggcaagtatctgctcaacaaccccaatgctggcaacacgaacatcttccgttccaccgggaaccagaactttgaccttaattcgtggagcccgtggaccgtccccaagctgtcttag

SEQ ID NO: 12 - protein, 474 aa, Chaetomium globosum
MLSPALASSLLVVLGAAGAHAQQRQALWGQCGGSGWNGPTACVDGAYCFQQNQWYHQCIPGSDRSPSPTNPPQQSSTLTTSRVTVAPPTSTSPAPGGGGTTCPATPSGQGSPVANALNDPFTFHGGGKVSSKADWTCRQAEISKLLQQYELGTLPPKPSSVTASFSGSTLSISVSEGGKSISFSVSINNRPSSSGPHPAIINFGTFGGLPVPAGVATINFNNDEIAQQQDQSSRGKGKFYDIYGSGHSAGALTAWAWGVSRIIDALELTRAQTNIDPARIGVTGCSRNGKGAMVAGALEPRIALTLPEESGAGGSGCWRIATWQKNRGENVQESKQIVTENVWFSTNFNSQVNNVNALPFDHHMLAGLIAPRALYVMENTDMEWLGKVSTYGCMGIARKQWEALGALDSFGYSQVGGNSHCSFPSSQQGSELNAFIGKYLLNNPNAGNTNIFRSTGNQNFDLNSWSPWTVPKLS

SEQ ID NO: 13 - DNA, 705 bp, Penicillium purporogenum
atgcattccaagtttttcgcagcctctcttcttggccttggcgcagctgccatccccctcgagggggtcatggagaaacgcagctgtcctgcgatccacgttttcggtgcccgtgaaaccactgcctctccagggtatggctcctccagcaccgtcgtcaatggcgttctcagcgcttacccgggatccaccgccgaggcaatcaactacccagcttgcggtggacaatcttcatgcgggggcgccagctattccagctcagtcgctcagggtattgccgctgtggcatccgctgtgaactcattcaattctcagtgcccgagcaccaagatagtcttggtcggatactctcagggtggtgaaatcatggacgtggccctgtgcggcggcggtgatcccaaccagggatacaccaacaccgctgtacagctgtcctcatcggctgtcaacatggtcaaagcggccattttcatgggcgacccaatgttccgagcgggactttcgtatgaggttggaacttgcgcggctggcggtttcgaccaacgcccggctggcttctcttgcccctcggctgctaagatcaagtcttactgcgatgcttcggacccgtactgctgcaacggtagtaatgcggcgactcatcagggttatggatctgagtatggttctcaggcattggctttcgtcaagagcaagctgggttaa

SEQ ID NO: 14 - protein, 234 aa, Penicillium purporogenum
MHSKFFAASLLGLGAAAIPLEGVMEKRSCPAIHVFGARETTASPGYGSSSTVVNGVLSAYPGSTAEAINYPACGGQSSCGGASYSSSVAQGIAAVASAVNSFNSQCPSTKIVLVGYSQGGEIMDVALCGGGDPNQGYTNTAVQLSSSAVNMVKAAIFMGDPMFRAGLSYEVGTCAAGGFDQRPAGFSCPSAAKIKSYCDASDPYCCNGSNAATHQGYGSEYGSQALAFVKSKLG

SEQ ID NO: 15 - DNA, 24 bp, Artificial Sequence
  Single strand DNA oligonucleotide
tggtgctagtcgggtattctcaag

SEQ ID NO: 16 - DNA, 24 bp, Artificial Sequence
  Single strand DNA oligonucleotide
tagatgcactaggacacacgaacc

SEQ ID NO: 17 - DNA, 24 bp, Artificial Sequence
  Single strand DNA oligonucleotide
caattccttcaactcgcagtgtcc

SEQ ID NO: 18 - DNA, 24 bp, Artificial Sequence
  Single strand DNA oligonucleotide
gcgtcgcaatagctcttgatcttg

SEQ ID NO: 19 - DNA, 24 bp, Artificial Sequence
  Single strand DNA oligonucleotide
gttgctccaagaccgttgataagc

SEQ ID NO: 20 - DNA, 24 bp, Artificial Sequence
  Single strand DNA oligonucleotide
agagtccgtttgtctcgaagatcg

SEQ ID NO: 21 - DNA, 1614 bp, Arabidopsis thaliana
gaagcttaacttgttgtttcataagttttaaaatttattatctaattatctacttttatgtgttctagagcaaagtgctaaatgtatatatacttagatgttgtgttgtaatccaatgtcaatataatcaatgatttagctatttgtaaacatactaaatagtattccaccaaaaaaaaaacatactaaacagtaaacaaacagcaaaaacaaaatccacatgtcctaaaagatagtctgattttcgttcataatgctctggtttttgaaagataataattgtgttgtatgagtgtatgacaaatattcattggtttgagaagttaacaaaatttggtggctacaaatggtttcctattcgagttgggtccattatcccttggcgtgtacggaaataatacctacccatcataatctgatcaaagatgaggtagtctttaaataaattttgcggcttatatcaatctttatgtactataaactgtgaactttttgttcttcaggacttccacatcattgcccaatccggttataccttcgctagttaatatgttaattaacattaaattaaagagctaacatttcttaggtagtaaaatagaagttttgaactactatactactaacatgtgaaaatactttagtcacaaatatgacaatatacaaatttattggaatgcaaattcttgaatttcaattgtttgaaaattatatatttctacataacaattctttataaactaaaaatattaattttccatggctatgcgttatacgtatatgtcaaatatttttattatttatataattttacgataaattagtactccactttactatattactcaacactaaaagacctctttaactccgcctaacatagatatgttttcttttgaatgtttcggttaaacatgacagagatttgttttcttgctttcgctcaatacatatttgtgctcctttagaaaagtagtatttcctaacaatccaacattttcatatttattatatcttttaaatattatcatggttctttttctttcgtcatgtttggcctctttaaaataattcttgaattgtatgagcattaatccaataacgtcctgatcccaaaaacctcatattaggtttgagagtccgaaaatatacttttcacataaagcacctaaggtgtcatactttaacaacttcacaaaatatgcaaaatttgtcattgtcactttgagatgtaagtttttttttacatgcaaatagattgagtctctttacgtgtaaattcatttaataaaattgtatggaatatctatttatatcatatatttctaacatatatataaatatctatacaaaaatacgactttttggcacattgtaattagaaaaatccacaagaaacagaaaaaagaaacaccaaatacaacgaaattgaagaaattattataaatttgaattggcttaacatctcttaagagtcaacaaggtaaaggattaattagtagtcttcatcaatctttctccaccttcttctattccttaatctccactttatctcccaaacccgaaaactcctcttcaccaacttaaaccctattaactaatcccaacaatcag

SEQ ID NO: 22 - protein, 24 aa, Artificial Sequence
  Arabidopsis endoglucanase cel1 signal peptide
MARKSLIFPVILLAVLLFSPPIYS

SEQ ID NO: 23 - protein, 20 aa, Artificial Sequence
  Arabidopsis Expansin-like A1 signal peptide
MGSFLFLIVVIFLFSSSVNA

SEQ ID NO: 24 - protein, 21 aa, Artificial Sequence
  Arabidopsis  Xyloglucan endotransglucosylase/hydrolase protein 22 signal peptide
MAITYLLPLFLSLIITSSVSA

SEQ ID NO: 25 - protein, 34 aa, Artificial Sequence
  Arabidopsis Pectinesterase/pectinesterase inhibitor 18, signal peptide
MSNSNQPLLSKPKSLKHKNLCLVLSFVAILGSVA

SEQ ID NO: 26 - protein, 26 aa, Artificial Sequence
  Arabidopsis extensin-like protein 1 signal peptide
MLFPPLRSLFLFTLLLSSVCFLQIKA

SEQ ID NO: 27 - protein, 21 aa, Artificial Sequence
  Arabidopsis  Laccase-15 signal peptide
MSHSFFNLFLISLFLYNNCIA

SEQ ID NO: 28 - protein, 27 aa, Artificial Sequence
  Populus alba Endo-1,4-beta glucanase signal peptide
MANATTFSLMLQFFFVTFCCLSYFSFA

SEQ ID NO: 29 - DNA, 828 bp, Arabidopsis thaliana
tgtctttggactgagagatcttaaaagagaatgtaaattactttaaacacggaataatggacaaaagccgtgatcaatgacttttcaagtcttaaccaaacctataactcatccattgtttgttttttctacatatttcttcacataaaattggatgatttagaatctttcagagtgttcacactccaacagattattatccacaatgttatggttacatttagagatatataacaatgttcatttcatcgttgctaatgacataaaacgatcaaaaactgaatcatagtacttcttttacagtgatctcaaatatattaatcgctaatcaatgaattatgtcacctataattgtcgtattaccaacaactataaaacatatatataaaaaattgttgtcgttaactagttgttgatagtggccactctaaaacgatcatgacctactacggaagttataactagtcaacgttggacgttagcaaggcccaatggacattaactcagcccataatagcacgcgccttgtgatgtgcaccagtttccgtctttggtcgttgaattcaaggaaaaaaaaagtacatcacaagcaatttcttacttatctgtgacttgaagctatttctccaatttcgttttccatcgacactctatttcattttcaccattcacgtcttccttctgaataaaataaaccctaaaacctaataccgaagtaaactcgtcaaccactgcgcccatgacctcccaacgatactcttcccttatattcttcctcttccttcttcctttctgcgatccaaaccttcaaacacatctccggtagat

SEQ ID NO: 30 - DNA, 1121 bp, Arabidopsis thaliana
agatcttttagttgtttgctttgtatgttgcgaacagtttgattctgtttttctttttcctttttttgggtaattttcttataacttttttcatagtttcgattatttggataaaattttcagattgaggatcattttatttatttattagtgtagtctaatttagttgtataactataaaattgttgtttgtttccgaatcataagttttttttttttttggttttgtattgataggtgcaagagactcaaaattctggtttcgatgttaacagaattcaagtagctgcccacttgattcgatttgttttgtatttggaaacaaccatggctggtcaaggcccagcccgttgtgcttctgaacctgcctagtcccatggactagatctttatccgcagactccaaaagaaaaaggattggcgcagaggaattgtcatggaaacagaatgaacaagaaagggtgaagaagatcaaaggcatatatgatctttacattctctttagcttatgtatgcagaaaattcacctaattaaggacagggaacgtaacttggcttgcactcctctcaccaaaccttaccccctaactaattttaattcaaaattactagtattttggccgatcactttatataataagataccagatttattatatttacgaattatcagcatgcatatactgtatatagttttttttttgttaaagggtaaaataataggatccttttgaataaaatgaacatatataattagtataatgaaaacagaaggaaatgagattaggacagtaagtaaaatgagagagacctgcaaaggataaaaaagagaagcttaaggaaaccgcgacgatgaaagaaagacatgtcatcagctgatggatgtgagtgatgagtttgttgcagttgtgtagaaatttttactaaaacagttgtttttacaaaaaagaaataatataaaacgaaagcttagcttgaaggcaatggagactctacaacaaactatgtaccatacagagagagaaactaaaagcttttcacacataaaaaccaaacttattcgtctctcattgatcaccgttttgttctctcaagatcgctgctaatctccggccgtccct

SEQ ID NO: 31 - DNA, 1991 bp, Eucalyptus grandis
ggaagcttcccgggtgagctcaagcctcatcaacgctagaccacaacgagctcttcccttgcttggccgatcactaggcattatcgtggttgaccactagcgaggaagatggaagaaggaagaagaataaaggaaaaaggaaaaagaaaaaataattataattataattttgattttaaatttttatttataaggtcgatggggagggagagtggagagggcatgagtctagtgggttggaaattgtttgaaactcaacttacttagacctgtttattaatgtcttcaaatttaaaatccatttaaggtaggccattcaaaattaaggtgatccatatgtaactcatttatgtttgaaatggaacatatatggatttaagatccattttcataggtctatccacgattaacttttttttatttgtcctctatttaaacatattggactggaaatcaaattaacaattccgttatattgaactaacaaaaagataatatcaaatattttatgtatccgagactaaattgaatatttacaaaaaaaatgtaaggatgacattgaataaattaaaaacttaaaaatgatattataagtcggggctaaaattcagagatcatttttgtcattcttcctttttatcttcccagaaaatggtagatcatcgccacaaattacctccaaatgaccaaaaagaaactcccatctcctccagctcttctctttccccaaccgccgacgatcccgccgtcccccacacatgggcgaaacaattaaaaataccctttttttataaatatctcgtgcttctgcttcccttctctgcgatttttaacagggaaaaattgagtcaatgatgggggtggttgctccgcgttttctttggcaaacgtcatttcagtggttagctgatttagacgggatgcaaaaggcaaggagagagcgatgatgattggtagggatgtcttaacgtcagatctcgccagttcctctcgttgccgtttgatctgtcggtggaggcttcggtcctggaaataccaattacccttccagggcagctgctgctccgtcgctttcgtcgctcccaagcaatttccttccccttcccccttttgcaaagtcacgaaattaggaattcccaactacagctggactccgacactcggacagcgggggagtaaaacggaaaaaacaagaaaagattgaattttttttctccttcttttacaaactctaactaaagagattaaaataaagaaacggaggaattatatacacattgccgctagtgggtacatccccatcacatcgcatctacaaagcgcgagcgagagaaccagaggagagacagctagcgtttccccgcacaccactctctctctctctctctctctgctcatcctcttctctctctcagctcgggtcagtttcgatctgcattttttcatgctttccctctgggttcggttcggttcggttcggttcggttctgttggattcgattcgatggtgagttgaagaaagtgctcttctttgtgcaggaactgagcgtttcgcctaccgtcctccgtcgttctatccggtcaagatcggattttgaggtgagtgcactgcttaatttcccattttctcccgggggtcatgatctgtgattgccgaagtaggacggataatctggaaggtttggatctcgtcactcgagattcgactttgttctttgactggtgaacaagggggaaatgagatttattcagtcaagctgaagaatcgttgcctgcttatgcggtccttatagaagctagtccaagtctctaaatttcgtccgcaattgtcgttcaattggagcgatcttgggcttgtggaaggagaataacttatttttcgcaactttttcaggaatttactcacggatctgtgtttttactggaaaacaagttgcttctgaatgcaacgctagagatctctacagcttctgctaatgccactctagacc

SEQ ID NO: 32 - DNA, 664 bp, Eucalyptus grandis
aaaaaaagaaaaagaaaaagaaaggaaagaaatgggggaagaaaatgggggaagagaaaggagaaaaaaaggaaagacgcgacagccagaacccaaaaatcccagcccggaatcgccattaaagcgcccttaaattaattccccatctcctcccccaaaaaataaaactgaaatcccctcccctctctctctctctctctactttctctcactaccaactcgaccttcctcctcctgtttttttttttttttgggtttttgtcgtggccggtgggggcacccagcccagcgccaccaccacattgtggtccccacctccttctctctctatgactgacttttctcattctctctctctatctcttctggagtctcgattagattggattcactcgtacaacaagcaccagcacatctctgcatccatctgctcctcctttctcactccttccccacttcccttcccttccctgtcacgcctctcccccctctctctctagacgctcgcgaatacgcaggcgagacccatttcctcccttcctttctctctgtgtgaatctacccgtctaaaaaaggctgtccgcagcacattgatcgagatcgagagcgcagcagagcatcccccgctcgacaagcattctcccccgccagatcgggcgctgca

(19) United States
(12) Patent Application Publication
(10) Pub. No.: US 2013/0227724 A1
(43) Pub. Date: Aug. 29, 2013

(54) TRANSGENIC PLANTS WITH IMPROVED SACCHARIFICATION YIELDS AND METHODS OF GENERATING SAME

(71) Applicants: Miron Abramson, Karmei Yosef (IL); Ziv Shani, Mazkere Batia (IL); Oded Shoseyov, Karmei Yosef (IL)

(72) Inventors: Miron Abramson, Karmei Yosef (IL); Ziv Shani, Mazkere Batia (IL); Oded Shoseyov, Karmei Yosef (IL)

(73) Assignee: Yissum Research Development Company of the Hebrew University of Jerusalem Ltd., Jerusalem (IL)

(21) Appl. No.: 13/883,046

(22) PCT Filed: Nov. 3, 2011
(86) PCT No.: PCT/IL11/00855

§ 371 Date: May 2, 2013

Related U.S. Application Data
(60) Provisional application No. 61/409,590, filed on Nov. 3, 2010.

(51) Int. Cl. C12N 15/82

(52) U.S. Cl. CPC C12N 15/8255; C12N 15/8243
USPC 800/260; 800/287; 800/298; 800/317.3; 435/165; 435/160; 435/140; 435/135; 435/320.1; 426/635; 426/615

(57) ABSTRACT

A method of engineering a plant having reduced acetylation in a cell wall is disclosed. The method comprising expressing in the plant cell wall at least one isolated heterologous polynucleotide encoding an acetylxylan esterase (AXE) enzyme under the transcriptional control of a developmentally regulated promoter specifically active in the plant cell wall upon secondary cell wall deposit, thereby engineering the plant having reduced acetylation in the cell wall.

### FIELD AND BACKGROUND OF THE INVENTION

The present invention, in some embodiments thereof, relates to transgenic plants expressing acetylxylan esterase (AXE) and/or glucuronoyl esterase (GE) and, more particularly, but not exclusively, to the use of same in various applications such as for biomass conversion (e.g. biofuels, hydrogen production), for feed and food applications, and for pulp and paper industries.

Natural resources and environmental quality are in constant decline in line with the rapid growth of the world's population. Current methods of energy consumption based primarily on fossil fuels, are considered environmentally hazardous and contribute to global warming. To address this growing concern, interest has increased in producing fuels from renewable resources, particularly those derived from plant biomass. To date, most ethanol fuel has been generated from corn grain or sugar cane, also referred to as “first generation” feedstock. Bioconversion of such crops to biofuel competes with food production for land and water resources, has a high feedstock cost and replaces only a small proportion of fossil fuel production. The main challenges associated with development of “second generation” biomass-derived biofuels include maximization of biomass yield per hectare per year, maintenance of sustainability while minimizing agricultural inputs and prevention of competition with food production.

With these considerations in mind, much focus has been placed on conversion of lignocellulosic biomass to fermentable sugars. Ultimately, lignocellulosic-derived ethanol has the potential to meet most of the global transportation fuel needs with much less impact on food supply, with lower agricultural inputs and less net carbon dioxide emissions compared to fossil fuels. However, due to the complex structure of plant cell walls, cellulosic biomass is more difficult to break down into sugars than starch found in the first generation biomass. Lignocellulosic biomass feedstock is made up of complex structures mainly comprising cellulose, hemicellulose and lignin designed by nature to provide structural support and resist breakdown by various organisms and their related enzymes.

The amount of each component, the ratio between them and the type of the hemicellulose is largely dependent on the feedstock type.

Currently, conversion of lignocellulosic biomass to bioethanol utilizes a three step process involving a pretreatment stage (e.g. heat/acid-based pretreatment) followed by saccharification of cellulose and hemicellulose to simple sugars via hydrolysis and finally fermentation of the free sugars to ethanol or butanol. Additional conversion pathways, such as those which utilize intermediate degradation products, have also been contemplated. The pretreatment phase is characterized by removal of crosslinking bonds between the matrix polysaccharides and lignin within the cell wall using toxic solvents and high energy inputs [i.e. the reaction conditions can utilize for example up to 3% sulfuric acid, between 120° C.-200° C. and pressure of 3-15 atm; Wyman C E et al., Bioresour Technol (2005) 96 (18):1959-1966]. Aside from the costliness of methods such as these, the pre-treatment phase produces toxic byproducts such as acetic acid and furfurals that subsequently inhibit hydrolytic enzymes and fermentation during later stages of the processing.

The degree of lignification and cellulose crystallinity are the most significant factors believed to contribute to the recalcitrance of lignocellulosic feedstock to chemicals or enzymes. Therefore, the current production processes involve large amounts of heat energy and concentrated acids that cause the cell wall to swell, thereby enabling removal of lignin and/or enabling solubilization of some hemicelluloses rendering the cellulosic polysaccharides more accessible to the saccharification process.

Current transgenic strategies to generate lignocellulose crops more amenable to saccharification have focused on modification of the cell wall lignin components. For example, manipulation of lignin biosynthesis resulted in lowered lignin and increased polysaccharide-degradability but also had a detrimental effect on the mechanical support, disease resistance and water transport of the engineered plants [Halpin C et al., Tree Genetics & Genomes (2007) 3 (2):101-110; Pedersen J F et al., Crop Science (2005) 45 (3):812-819].

Additional background art includes U.S. Application No. 20070250961, U.S. Pat. No. 7,666,648, PCT Application No. WO2009033071, U.S. Application No. 20100017916, U.S. Application No. 20100031400, U.S. Application No. 20100043105, PCT Application No. WO2009042846, PCT Application No. WO2009132008, PCT Application No. WO2009155601, U.S. Application No. 20100031399 and PCT Application No. WO2009149304.

### SUMMARY OF THE INVENTION

According to an aspect of some embodiments of the present invention there is provided a method of engineering a plant having reduced acetylation in a cell wall, the method comprising expressing in the plant cell wall at least one isolated heterologous polynucleotide encoding an acetylxylan esterase (AXE) enzyme under the transcriptional control of a developmentally regulated promoter specifically active in the plant cell wall upon secondary cell wall deposit, thereby engineering the plant having reduced acetylation in the cell wall.

According to an aspect of some embodiments of the present invention there is provided a method of engineering a plant having reduced acetylation in a cell wall, the method comprising expressing in the plant cell wall at least one isolated heterologous polynucleotide encoding an acetylxylan esterase (AXE) enzyme, wherein the AXE enzyme is selected from the group consisting of SEQ ID NO: 2, SEQ ID NO: 4, SEQ ID NO: 6 and SEQ ID NO: 14, thereby engineering the plant having reduced acetylation in the cell wall.

According to an aspect of some embodiments of the present invention there is provided a method of engineering a plant having reduced lignin hemicellulose ester crosslinks in a cell wall, the method comprising expressing in the plant cell wall at least one isolated heterologous polynucleotide encoding a glucuronoyl esterase (GE) enzyme under the transcriptional control of a developmentally regulated promoter specifically active in the plant cell wall upon secondary cell wall deposit, thereby engineering the plant having reduced lignin hemicellulose ester crosslinks in the cell wall.

According to an aspect of some embodiments of the present invention there is provided a method of engineering a plant having reduced lignin hemicellulose ester crosslinks in a cell wall, the method comprising expressing in the plant cell wall at least one isolated heterologous polynucleotide encoding a glucuronoyl esterase (GE) enzyme, wherein the GE enzyme is selected from the group consisting of SEQ ID NO: 8, SEQ ID NO: 10 and SEQ ID NO: 12, thereby engineering the plant having reduced lignin hemicellulose ester crosslinks in the cell wall.

According to an aspect of some embodiments of the present invention there is provided a genetically modified plant expressing a heterologous polynucleotide encoding an acetylxylan esterase (AXE) enzyme under the transcriptional control of a developmentally regulated promoter specifically active in the plant cell wall upon secondary cell wall deposit.

According to an aspect of some embodiments of the present invention there is provided a genetically modified plant expressing a heterologous polynucleotide encoding an acetylxylan esterase (AXE) enzyme as set forth in SEQ ID NOs: 2, 4, 6 or 14.

According to an aspect of some embodiments of the present invention there is provided a genetically modified plant expressing a heterologous polynucleotide encoding a glucuronoyl esterase (GE) enzyme under the transcriptional control of a developmentally regulated promoter specifically active in the plant cell wall upon secondary cell wall deposit.

According to an aspect of some embodiments of the present invention there is provided a genetically modified plant expressing a heterologous polynucleotide encoding a glucuronoyl esterase (GE) enzyme as set forth in SEQ ID NOs: 8, 10 or 12.

According to an aspect of some embodiments of the present invention there is provided a genetically modified plant co-expressing a heterologous polynucleotide encoding an acetylxylan esterase (AXE) enzyme and a heterologous polynucleotide encoding a glucuronoyl esterase (GE) enzyme under the transcriptional control of a developmentally regulated promoter specifically active in the plant cell wall upon secondary cell wall deposit.

According to an aspect of some embodiments of the present invention there is provided a genetically modified plant co-expressing a heterologous polynucleotide encoding an acetylxylan esterase (AXE) enzyme as set forth in SEQ ID NOs: 2, 4, 6 or 14 and a heterologous polynucleotide encoding a glucuronoyl esterase (GE) enzyme as set forth in SEQ ID NOs: 8, 10 or 12.

According to an aspect of some embodiments of the present invention there is provided a plant system comprising: (i) the first genetically modified plant of claim 13 or 14; and (ii) the second genetically modified plant of claim 15 or 16.

According to an aspect of some embodiments of the present invention there is provided a method of producing a plant having reduced acetylation and reduced lignin hemicellulose ester crosslinks in a cell wall, the method comprising: (a) expressing in a first plant a heterologous polynucleotide encoding an acetylxylan esterase (AXE) enzyme under the transcriptional control of a developmentally regulated promoter specifically active in the cell wall upon secondary cell wall deposit; (b) expressing in a second plant a heterologous polynucleotide encoding a glucuronoyl esterase (GE) enzyme under the transcriptional control of a developmentally regulated promoter specifically active in the cell wall upon secondary cell wall deposit; and (c) crossing the first plant and the second plant and selecting progeny expressing the acetylxylan esterase (AXE) enzyme and the glucuronoyl esterase (GE) enzyme, thereby producing the plant having the reduced acetylation and the reduced lignin hemicellulose ester crosslinks in the cell wall.

According to an aspect of some embodiments of the present invention there is provided a method of producing a plant having reduced acetylation and reduced lignin hemicellulose ester crosslinks in a cell wall, the method comprising: (a) expressing in a first plant a heterologous polynucleotide encoding an acetylxylan esterase (AXE) enzyme as set forth in SEQ ID NOs: 2, 4, 6 or 14; (b) expressing in a second plant a heterologous polynucleotide encoding a glucuronoyl esterase (GE) enzyme as set forth in SEQ ID NOs: 8, 10 or 12; and (c) crossing the first plant and the second plant and selecting progeny expressing the acetylxylan esterase (AXE) enzyme and the glucuronoyl esterase (GE) enzyme, thereby producing the plant having the reduced acetylation and the reduced lignin hemicellulose ester crosslinks in the cell wall.

According to an aspect of some embodiments of the present invention there is provided a food or feed comprising the genetically modified plant of claim 13, 14, 15, 16, 17, 18, 20 or 21.

According to an aspect of some embodiments of the present invention there is provided a method of producing a biofuel, the method comprising: (a) growing the genetically modified plant of any of claim 13, 14, 15, 16, 17, 18, 20 or 21, under conditions which allow degradation of lignocellulose to form a hydrolysate mixture; and (b) incubating the hydrolysate mixture under conditions that promote conversion of fermentable sugars of the hydrolysate mixture to ethanol, butanol, acetic acid or ethyl acetate, thereby producing the biofuel.

According to an aspect of some embodiments of the present invention there is provided a nucleic acid construct comprising a polynucleotide encoding a heterologous acetylxylan esterase (AXE) enzyme under the transcriptional control of a developmentally regulated promoter specifically active in the plant cell wall upon secondary cell wall deposit.

According to an aspect of some embodiments of the present invention there is provided a nucleic acid construct comprising a polynucleotide encoding a heterologous glucuronoyl esterase (GE) enzyme under the transcriptional control of a developmentally regulated promoter specifically active in the plant cell wall upon secondary cell wall deposit.

According to an aspect of some embodiments of the present invention there is provided a nucleic acid construct comprising a polynucleotide encoding a heterologous acetylxylan esterase (AXE) enzyme and a polynucleotide encoding a heterologous glucuronoyl esterase (GE) enzyme both under the transcriptional control of a developmentally regulated promoter specifically active in the plant cell wall upon secondary cell wall deposit.

According to an aspect of some embodiments of the present invention there is provided a nucleic acid construct comprising a polynucleotide encoding a heterologous acetylxylan esterase (AXE) enzyme as set forth in SEQ ID NO: 1 under the transcriptional control of a FRA8 promoter.

According to an aspect of some embodiments of the present invention there is provided a nucleic acid construct comprising a polynucleotide encoding a heterologous acetylxylan esterase (AXE) enzyme as set forth in SEQ ID NO: 13 under the transcriptional control of a FRA8 promoter.

According to an aspect of some embodiments of the present invention there is provided a nucleic acid construct comprising a polynucleotide encoding a heterologous glucuronoyl esterase (GE) enzyme as set forth in SEQ ID NO: 7 under the transcriptional control of a FRA8 promoter.

According to some embodiments of the invention, the method further comprises expressing in the plant an additional heterologous polynucleotide encoding a glucuronoyl esterase (GE) enzyme.

According to some embodiments of the invention, the method further comprises expressing in the plant an additional heterologous polynucleotide encoding an acetylxylan esterase (AXE) enzyme.

According to some embodiments of the invention, the additional heterologous polynucleotide is expressed under the transcriptional control of a developmentally regulated promoter specifically active in the plant cell wall upon secondary cell wall deposit.

According to some embodiments of the invention, the AXE enzyme is selected from the group consisting of SEQ ID NO: 2, SEQ ID NO: 4, SEQ ID NO: 6 and SEQ ID NO: 14.

According to some embodiments of the invention, the GE enzyme is selected from the group consisting of SEQ ID NO: 8, SEQ ID NO: 10 and SEQ ID NO: 12.

According to some embodiments of the invention, the isolated heterologous polynucleotide is expressed in a tissue specific manner.

According to some embodiments of the invention, the tissue is selected from the group consisting of a stem and a leaf.

According to some embodiments of the invention, the tissue comprises a xylem or a phloem.

According to some embodiments of the invention, the heterologous polynucleotide is expressed under the transcriptional control of a developmentally regulated promoter specifically active in the plant cell wall upon secondary cell wall deposit.

According to some embodiments of the invention, the AXE enzyme is as set forth in SEQ ID NOs: 2, 4, 6 or 14.

According to some embodiments of the invention, the GE enzyme is as set forth in SEQ ID NOs: 8, 10 or 12.

According to some embodiments of the invention, the heterologous polynucleotide encoding the AXE enzyme is as set forth in SEQ ID NOs: 1, 3, 5 or 13 and the heterologous polynucleotide encoding the GE enzyme is as set forth in SEQ ID NOs: 7, 9 or 11.

According to some embodiments of the invention, the plant comprises reduced covalent links between a hemicellulose and a lignin in a cell of the plant as compared to a non-transgenic plant of the same species.

According to some embodiments of the invention, the plant comprises reduced acetylation in a cell of the plant as compared to a non-transgenic plant of the same species.

According to some embodiments of the invention, the plant is selected from the group consisting of a corn, a switchgrass, a sorghum, a miscanthus, a sugarcane, a poplar, a pine, a wheat, a rice, a soy, a cotton, a barley, a turf grass, a tobacco, a bamboo, a rape, a sugar beet, a sunflower, a willow, a hemp, and an eucalyptus.

According to some embodiments of the invention, the conditions comprise less pretreatment chemicals then required by a non-transgenic plant of the same species.

According to some embodiments of the invention, the polynucleotide encoding the AXE enzyme is as set forth in SEQ ID NOs: 1, 3, 5 or 13.

According to some embodiments of the invention, the polynucleotide encoding the GE enzyme is as set forth in SEQ ID NOs: 7, 9 or 11.

According to some embodiments of the invention, the nucleic acid construct further comprises a nucleic acid sequence encoding a signal peptide capable of directing AXE or GE expression in a plant cell wall.

According to some embodiments of the invention, the heterologous polynucleotide encoding the AXE enzyme or the GE enzyme is conjugated to a nucleic acid sequence encoding a signal peptide capable of directing AXE or GE expression in a plant cell wall.

According to some embodiments of the invention, the signal peptide is selected from the group consisting of an Arabidopsis endoglucanase cell signal peptide, an Arabidopsis thaliana Expansin-like A1, an Arabidopsis thaliana Xyloglucan endotransglucosylase/hydrolase protein 22, an Arabidopsis thaliana Pectinesterase/pectinesterase inhibitor 18, an Arabidopsis thaliana extensin-like protein 1, an Arabidopsis thaliana Laccase-15 and a Populus alba Endo-1,4-beta glucanase.

According to some embodiments of the invention, the signal peptide comprises a Arabidopsis endoglucanase cell signal peptide.

According to some embodiments of the invention, the promoter is selected from the group consisting of 4c1, CesA1, CesA7, CesA8, IRX3, IRX4, IRX10, DOT1 and FRA8.

According to some embodiments of the invention, the promoter comprises FRA8.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an illustration of acetylated xylan modified by acetylxylan esterases (AXEs).

FIGS. 2A-B is an illustration of expression of fungal AXE in plants cell walls leading to enhanced xylan solubility. FIG. 2A depicts wild type cellulose microfibrils containing tightly packed matrix of cellulose and xylan, with limited access to hydrolysing enzymes; and FIG. 2B depicts expression of AXE increasing xylan solubility following pretreatment, exposing cellulose to hydrolytic enzymes.

FIG. 3 is an illustration of glucuronoyl esterase (GE) de-esterifying the chemical bond between lignin and 4-O-Me-GlcA residue of Glucuronoxylan (GX).

FIGS. 4A-D are schematic illustrations of AXE and GE vectors which can be generated according to some embodiments of the invention and used for plant transformation.

FIGS. 5A-D depict PCR analysis of expression of heterologous AXE/GE in transgenic tobacco plants. FIG. 5A illustrates PCR results for plants transformed with the FRA8::AXEI vector; FIG. 5B illustrates PCR results for plants transformed with the FRA8::AXEII vector; FIG. 5C illustrates PCR results for plants transformed with the FRA8::GE vector; and FIG. 5D illustrates PCR results for plants transformed with the 35S::AXEII vector. Numbers represent independent event, +control represents positive control, w.t=wild type plant (a non-transformed plant grown under the same growth conditions).

FIGS. 6A-H depict RT-PCR products of AXEs and GE genes in wild type (WT) and transgenic plants. Representative independent lines are shown. FIG. 6A illustrates results for plants transformed with the FRA8::AXEI vector; FIG. 6B illustrates results for plants transformed with the FRA8::AXEII vector; FIG. 6C illustrates results for plants transformed with the FRA8::GE vector; and FIG. 6D illustrates results for plants transformed with the 35S::AXEII vector. W.T=wild type; numbers represent independent lines.

FIG. 7 depicts acetylxylan esterase activity (pNP-acetyl was used as a substrate) in 4-week old transgenic plants expressing different AXEs and wild types (WT).

FIG. 8 depicts quantitative analysis of acetyl groups released from the cell walls of 4-week old stems of the wild type (WT) and AXE plants. Cell wall material (CWM) of the stems was treated with NaOH and the released acetyl groups were analyzed. Data represents means of two separate assays.

FIG. 9 depicts saccharification of biomass from 4-week old stems of tobacco plants expressing the different AXEs, GE or wild type plants (WT). Reducing sugars released from 1 mg of biomass after hot water pretreatment followed by 24-h enzymatic hydrolysis were measured using the DNS assay.

### DESCRIPTION OF SPECIFIC EMBODIMENTS OF THE INVENTION

The present invention, in some embodiments thereof, relates to transgenic plants expressing acetylxylan esterase (AXE) and/or glucuronoyl esterase (GE) and, more particularly, but not exclusively, to the use of same in various applications such as for biomass conversion (e.g. biofuels, hydrogen production), for feed and food applications, and for pulp and paper industries.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The present inventors have uncovered new methods of engineering plants by modifying the plant cell wall and increasing cellulose accessibility within the lignocellulosic biomass of plants. According to the present teachings, the plant's phenotype is altered such that the plant cell wall is modified during plant growth. This optimized modification allows for the production of plants which maintain sufficient lignocellulose integrity to provide for upright plants capable of high density cultivation in a field. Moreover, the present invention enables the production of plants optimized for the industrial saccharification process without adversely affecting the mechanical fitness of the engineered plants.

As is illustrated hereinbelow and in the Examples section which follows, the present inventors have generated nucleic acid constructs comprising the polynucleotide sequences of the acetylxylan esterase enzyme (AXE, e.g. SEQ ID NOs: 1 or 13) or the glucuronoyl esterase enzyme (GE, e.g. SEQ ID NO: 7) fused to a developmentally specific promoter capable of directing expression of the enzymes upon secondary cell wall development (e.g. FRA8, see Example 1). In order to localize the AXE and GE enzymes to the cell wall, the nucleic acid constructs were further generated to include an in frame cell wall specific signal peptide (e.g. Arabidopsis endoglucanase cell signal peptide, SEQ ID NO: 22, see Example 1). The present inventors have shown that expression of these nucleic acid constructs in tobacco plants led to generation of upright transgenic plants (data not shown) comprising active AXEI and AXEII proteins (Example 4). The present inventors have further shown a 50% reduction in acetic acid release in cell wall material (CWM, see Example 5) and an improvement of saccharification efficiency of 5% to 40% in plants expressing AXE or GE compared with wild type plants (Example 6). These results were superior to transgenic plants expressing AXE enzymes under the control of a constitutive promoter (35S).

Thus, according to one aspect of the present invention there is provided a method of engineering a plant having reduced acetylation in a cell wall, the method comprising expressing in the plant cell wall at least one isolated heterologous polynucleotide encoding an acetylxylan esterase (AXE) enzyme under the transcriptional control of a developmentally regulated promoter specifically active in the plant cell wall upon secondary cell wall deposit, thereby engineering the plant having reduced acetylation in the cell wall of the plant.

According to another aspect, there is provided a method of engineering a plant having reduced acetylation in a cell wall of the plant, the method comprising expressing in the plant cell wall at least one isolated heterologous polynucleotide encoding an acetylxylan esterase (AXE) enzyme, wherein the AXE enzyme is selected from the group consisting of SEQ ID NO: 2, SEQ ID NO: 4, SEQ ID NO: 6 and SEQ ID NO: 14, thereby engineering the plant having reduced acetylation in the cell wall.

According to another aspect there is provided a method of engineering a plant having reduced lignin hemicellulose ester crosslinks in a cell wall, the method comprising expressing in the plant cell wall at least one isolated heterologous polynucleotide encoding a glucuronoyl esterase (GE) enzyme under the transcriptional control of a developmentally regulated promoter specifically active in the plant cell wall upon secondary cell wall deposit, thereby engineering the plant to reduce lignin hemicellulose ester crosslinks in the cell wall.

According to another aspect there is provided a method of engineering a plant having reduced lignin hemicellulose ester crosslinks in a cell wall, the method comprising expressing in the plant cell wall at least one isolated heterologous polynucleotide encoding a glucuronoyl esterase (GE) enzyme, wherein the GE enzyme is selected from the group consisting of SEQ ID NO: 8, SEQ ID NO: 10 and SEQ ID NO: 12, thereby engineering the plant to reduce lignin hemicellulose ester crosslinks in the cell wall.

As used herein the term “plant” refers to whole plants, plant components (e.g. e.g., cuttings, tubers, pollens), plant organs (e.g., leaves, stems, flowers, roots, fruits, seeds, branches, etc.) or cells isolated therefrom (homogeneous or heterogeneous populations of cells).

As used herein, the phrase “genetically modified plant” refers to a plant in which one or more of the cells of the plant is stably or transiently transformed with an exogenous polynucleotide sequence introduced by way of human intervention. Transgenic plants typically express DNA sequences, which confer the plants with characters different from that of native, non-transgenic plants of the same strain.

As used herein the phrase “isolated plant cells” refers to plant cells which are derived from disintegrated plant cell tissue or plant cell cultures.

Any commercially or scientifically valuable plant is envisaged in accordance with these embodiments of the invention. A suitable plant for use with the method of the invention can be any higher plant amenable to transformation techniques, including both monocotyledonous or dicotyledonous plants, as well as certain lower plants such as algae and moss. The term plant as used herein refers to both green field plants as well as plants grown specifically for biomass energy. Plants of the present invention, include, but are not limited to, alfalfa, bamboo, barley, beans, beet, broccoli, cabbage, canola, chile, carrot, corn, cotton, cottonwood (e.g. Populus deltoides), eucalyptus, hemp, hibiscus, lentil, lettuce, maize, miscanthus, mums, oat, okra, peanut, pea, pepper, potato, poplar, pine (pinus sp.), potato, rape, rice, rye, soybean, sorghum, sugar beet, sugarcane, sunflower, sweetgum, switchgrass, tomato, tobacco, turf grass, wheat, and willow, as well as other plants listed in World Wide Web (dot) nationmaster (dot) com/encyclopedia/Plantae.

Accordingly, plant families may comprise Alliaceae, Amaranthaceae, Amaryllidaceae, Apocynaceae, Asteraceae, Boraginaceae, Brassicaceae, Campanulaceae, Caryophyllaceae, Chenopodiaceae, Compositae, Cruciferae, Cucurbitaceae, Euphorbiaceae, Fabaceae, Gramineae, Hyacinthaceae, Labiatae, Leguminosae-Papilionoideae, Liliaceae, Linaceae, Malvaceae, Phytolaccaceae, Poaceae, Pinaceae, Rosaceae, Scrophulariaceae, Solanaceae, Tropaeolaceae, Umbelliferae and Violaceae.

The phrase “cell wall of the plant” as used herein refers to the layer that surrounds the plant cell membrane and provides plant cells with structural support and protection and typically acts as a filtering mechanism. The plant cell wall of the present teachings may comprise the primary cell wall and/or the secondary cell wall.

Normally the primary cell wall is composed predominantly of polysaccharides (e.g. cellulose, hemicellulose and pectin) together with lesser amounts of structural glycoproteins (hydroxyproline-rich extensins), phenolic esters (ferulic and coumaric acids), ionically and covalently bound minerals (e.g. calcium and boron), enzyme and proteins (e.g. expansins). The cellulose microfibrils are linked via hemicellulosic tethers to form the cellulose-hemicellulose network, which is embedded in the pectin matrix. The most common hemicellulose in the primary cell wall is xyloglucan.

The secondary walls of woody tissue and grasses are composed predominantly of cellulose, lignin and hemicellulose (xylan, glucuronoxylan, arabinoxylan, or glucomannan). The cellulose fibrils are embedded in a network of hemicellulose and lignin.

The present invention provides cell wall-modifying enzymes, specifically acetylxylan esterase (AXE) and glucuronoyl esterase (GE) enzymes, which may be used separately or combined to increase cellulose accessibility within the lignocellulosic biomass of a plant.

As used herein the term “acetylxylan esterase enzyme” (also termed acetyl xylan esterase or AXE) refers to the enzyme of the EC classification 3.1.1.72 that catalyzes the deacetylation of xylans and xylo-oligosaccharides in the cell wall of a plant. Exemplary AXE enzymes which may be used in accordance with the present invention are as set forth in SEQ ID NO: 2, SEQ ID NO: 4, SEQ ID NO: 6 and SEQ ID NO: 14.

As used herein the term “glucuronyl esterase enzyme” (also termed GE) refers to the enzyme of the EC classification 3.1.1.—that hydrolyzes the ester linkage between 4-O-methyl-D-glucuronic acid of glucuronoxylan and lignin alcohols in the covalent linkages connecting lignin and hemicellulose in plant cell walls. Exemplary GE enzymes which may be used in accordance with the present invention are as set forth in SEQ ID NO: 8, SEQ ID NO: 10 and SEQ ID NO: 12.

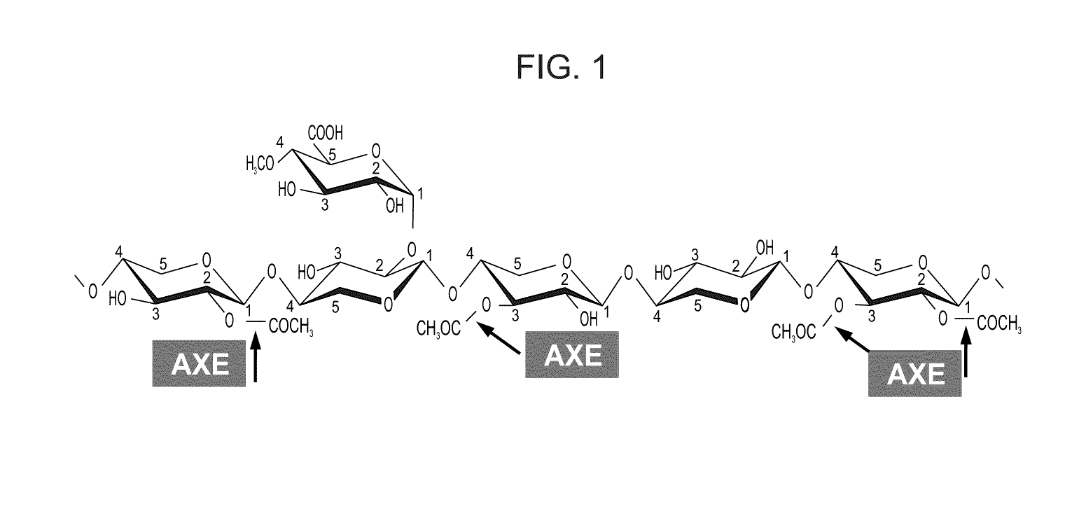

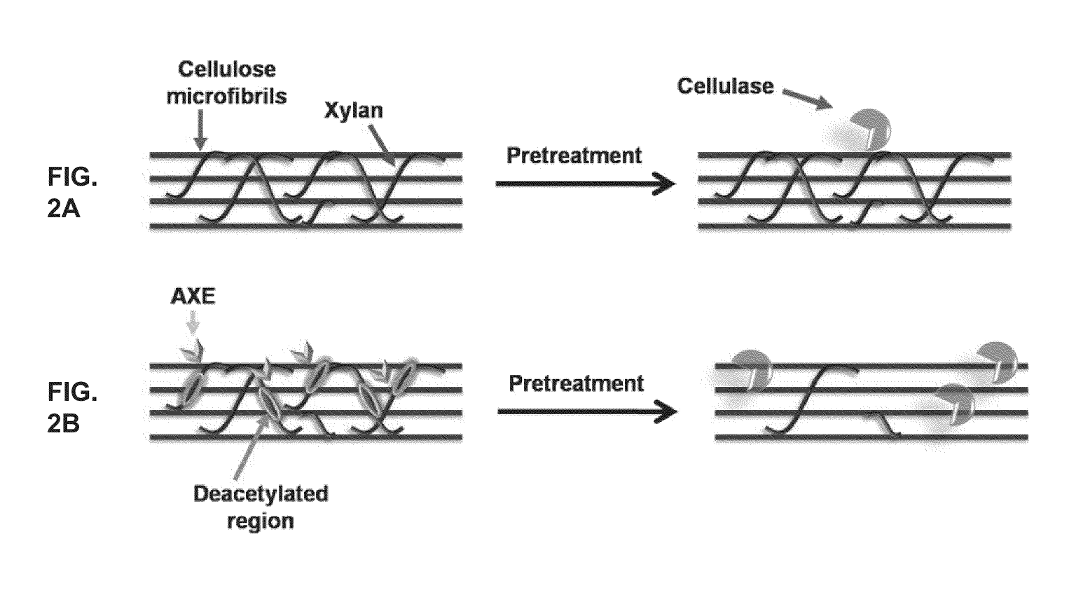

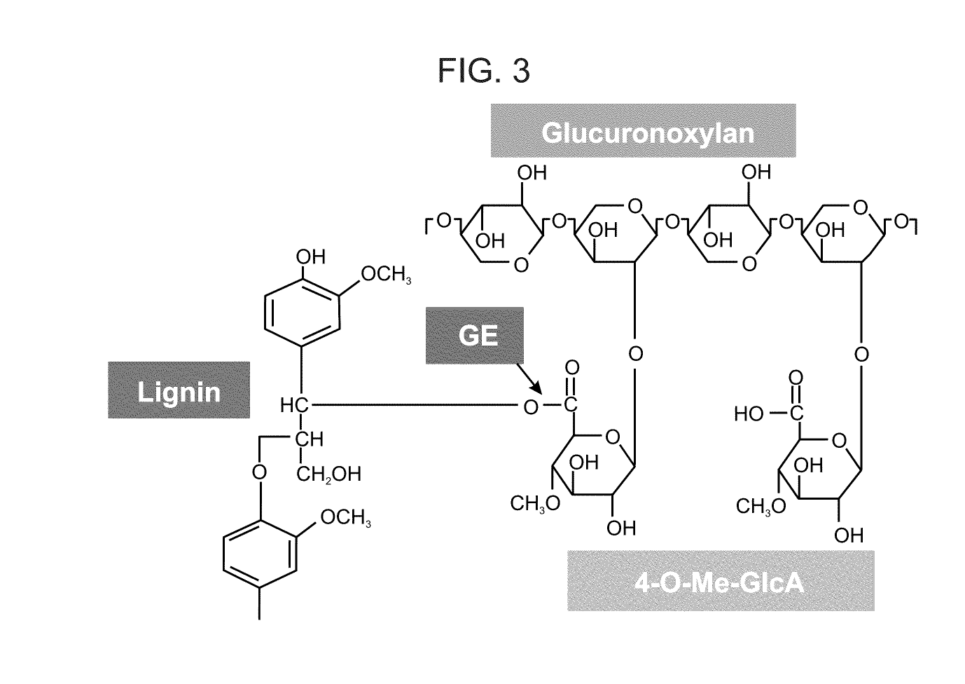

Lignocellulosic biomass is a complex substrate in which crystalline cellulose is embedded within a matrix of hemicellulose and lignin. Lignocellulose represents approximately 90% of the dry weight of most plant material with cellulose making up between about 30% to 50% of the dry weight of lignocellulose, and hemicellulose making up between about 20% and 50% of the dry weight of lignocellulose. Disruption and degradation (e.g., hydrolysis) of lignocellulose by lignocellulolytic enzymes, such as acetylxylan esterase enzyme (AXE, see FIGS. 1 and 2A-B) and glucuronoyl esterase enzyme (GE, see FIG. 3) as taught by the present invention, leads to: (1) reduction, by AXE, in acetylation of hemicuellulose residues in general and specifically xylan within the hemicelluloses; and (2) linkage break down, by GE, of hemicellulose-cellulose-lignin, hemicellulose-cellulose-pectin, glucoronoxylan-lignin, and combinations thereof, thereby leading to reduced cell wall crystallinity and/or to the formation of substances such as acetic acid, increasing polymer solubility, hydrolytic enzyme accessibility and consequently reducing pulping energy and bioconversion enzyme and energy input costs.

The term “cell wall acetylation” as used herein refers to the acetylation of xylans in the cell wall of a plant.

The term “reduced acetylation” as used herein refers to the deacetylation of xylans in the cell walls of the transgenic plant compared to those found in cell walls of a non-transgenic plant of the same species. Preferably, the reduction in the acetylation is a reduction of about 1%, about 2%, about 3%, about 4%, about 5%, about 6%, about 7%, about 8%, about 9%, about 10%, about 15%, about 20%, about 25%, about 30%, about 40%, about 50%, about 60%, about 70%, about 80%, about 90% or about 100% as compared to a non-transgenic plant of the same species. A transgenic plant with reduced acetylation typically comprises increased hydrolytic enzyme accessibility and consequently reduced pulping energy.

Methods of measuring acetylation in a plant cell wall may be effected using any method known to one of ordinary skill in the art, as for example, by first isolating cell walls from plant material, placing a sample of about 10 mg into a centrifuge tube fitted with e.g. gas-tight cap or lid, adding to each tube about 1 ml isopropanol/NaOH solution (at 4° C.), capping the tubes and mixing gently. Then the mixture can be left to stand for about 2 hours at room temperature followed by a centrifuge for about 10 min at 2,000×g (at room temperature). Supernatants can then be removed and placed in a small vial with a septum and immediately sealed. 15 μl of sample can then be injected into an HPLC system equipped with e.g. rezex RHM-Monosaccharide column and a 5 mM H₂SO₄ solvent system, set at a flow rate of about 0.6 ml/min and a temperature of 30° C. The refractive index detector used may be set at 40° C.

As used herein, the term “covalent links” refers to covalent bonds between a hemicellulose and a lignin found in the cell wall of a plant.

The term “reduced covalent links” as used herein refers to the number of covalent links between a hemicellulose and a lignin found in the cell wall of the transgenic plant compared to those found in the cell wall of a non-transgenic plant of the same species. Preferably, the reduction in the covalent links is a reduction of about 1%, about 2%, about 3%, about 4%, about 5%, about 6%, about 7%, about 8%, about 9%, about 10%, about 15%, about 20%, about 25%, about 30%, about 40%, about 50%, about 60%, about 70%, about 80%, about 90% or about 100% as compared to a non-transgenic plant of the same species. A transgenic plant with reduced covalent links between a hemicellulose and a lignin typically has enhanced separation of lignin and hemicellulose which result in more amendable feedstock for saccharification process and animal feed.

Methods of measuring reduced covalent links may be effected using any method known to one of ordinary skill in the art, as for example, by measuring the amount of ester linkages between lignin and hemicelluloses in the cell wall of a plant. An exemplary method comprises obtaining a FT-IR spectra of biomass sample on an FT-IR spectrophotometer using a KBr disk containing 1% finely ground samples. Subsequently, numerous scans are taken of each sample recorded from 4000 to 400 cm⁻¹ at a resolution of 2 cm⁻¹ in the transmission mode. A change in the peak at ˜1730 cm⁻¹ is typically correlated with the amount of uronic and ester groups or the ester binding of the carboxylic groups of ferulic and/or p-coumaric acids.

According to an aspect of the present invention, the method comprises expressing in the plant an additional heterologous polynucleotide encoding a glucuronoyl esterase (GE) enzyme. Exemplary GE enzymes which may be used in accordance with the present method are as set forth in SEQ ID NO: 8, SEQ ID NO: 10 and SEQ ID NO: 12.

According to an aspect of the present invention, the method comprises expressing in the plant an additional heterologous polynucleotide encoding an acetylxylan esterase (AXE) enzyme. Exemplary AXE enzymes which may be used in accordance with the present method are as set forth in SEQ ID NO: 2, SEQ ID NO: 4, SEQ ID NO: 6 and SEQ ID NO: 14.

Polynucleotides encoding the AXE and GE polypeptides contemplated herein also refer to functional equivalents of these enzymes. Methods of assaying AXE and GE activity are well known in the art and include, for example, measuring cell wall acetylation (i.e. for expression and activity of AXE), measuring the amount of ester linkages between lignin and hemicelluloses (i.e. for expression and activity of GE) or measuring saccharification yield and pulping efficiency of the transformed plants compared to non-transformed plants of the same type (as described in detail in Example 1 of the Examples section which follows).

Thus the polynucleotides described herein can encode polypeptides which are at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 75%, at least about 75%, at least about 75%, at least about 75%, say 100% identical or homologous to SEQ ID NOs: 2, 4, 6, 8, 10, 12, 14, as long as functionality is maintained.

Homology (e.g., percent homology) can be determined using any homology comparison software, including for example, the BlastP software of the National Center of Biotechnology Information (NCBI) such as by using default parameters.

Identity (e.g., percent homology) can be determined using any homology comparison software, including for example, the BlastN software of the National Center of Biotechnology Information (NCBI) such as by using default parameters.

As used herein the phrase “an isolated polynucleotide” refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

According to a preferred embodiment of this aspect of the present invention, the nucleic acid sequence is as set forth in SEQ ID NOs: 1, 3, 5, 7, 9, 11 or 13.

The AXE and/or GE enzymes described above can be expressed in the plant (e.g. in the cell wall thereof) from a stably integrated or a transiently expressed nucleic acid construct which includes polynucleotide sequences encoding the AXE enzyme, the GE enzymes or a construct co-expressing both the AXE and GE enzymes. The polynucleotide sequences are positioned under the transcriptional control of plant functional promoters. Such a nucleic acid construct (which is also termed herein as an expression construct) can be configured for expression throughout the whole plant, defined plant tissues or defined plant cells, or at define developmental stages of the plant. Such a construct may also include selection markers (e.g. antibiotic resistance), enhancer elements and an origin of replication for bacterial replication.

According to an embodiment of the present invention, the nucleic acid construct of the present invention comprises a polynucleotide encoding a heterologous acetylxylan esterase (AXE) enzyme under the transcriptional control of a developmentally regulated promoter specifically active in the plant cell wall upon secondary cell wall deposit.

According to an embodiment of the present invention, the nucleic acid construct of the present invention comprises a polynucleotide encoding a heterologous glucuronoyl esterase (GE) enzyme under the transcriptional control of a developmentally regulated promoter specifically active in the plant cell wall upon secondary cell wall deposit.

According to an embodiment of the present invention, the nucleic acid construct of the present invention comprises a polynucleotide encoding a heterologous acetylxylan esterase (AXE) enzyme and a polynucleotide encoding a heterologous glucuronoyl esterase (GE) enzyme both under the transcriptional control of a developmentally regulated promoter specifically active in the plant cell wall upon secondary cell wall deposit.

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells. The genetic construct can be an expression vector wherein the nucleic acid sequence is operably linked to one or more regulatory sequences allowing expression in the plant cells.

In a particular embodiment of the present invention the regulatory sequence is a plant-expressible promoter.

As used herein the phrase “plant-expressible” refers to a promoter sequence, including any additional regulatory elements added thereto or contained therein, is at least capable of inducing, conferring, activating or enhancing expression in a plant cell, tissue or organ, preferably a monocotyledonous or dicotyledonous plant cell, tissue, or organ.

It will be appreciated that constructs generated to include two expressible inserts (e.g. AXE and GE enzymes) preferably include an individual promoter for each insert, or alternatively such constructs can express a single transcript chimera including both insert sequences from a single promoter. In such a case, the chimeric transcript includes an IRES sequence between the two insert sequences such that the downstream insert can be translated therefrom.

Numerous plant functional expression promoters and enhancers which can be either tissue specific, developmentally specific, constitutive or inducible can be utilized by the constructs of the present invention, some examples are provided herein under.

As used herein in the specification and in the claims section that follows the phrase “plant promoter” or “promoter” includes a promoter which can direct gene expression in plant cells (including DNA containing organelles). Such a promoter can be derived from a plant, bacterial, viral, fungal or animal origin. Such a promoter can be constitutive, i.e., capable of directing high level of gene expression in a plurality of plant tissues, tissue specific, i.e., capable of directing gene expression in a particular plant tissue or tissues, inducible, i.e., capable of directing gene expression under a stimulus, or chimeric, i.e., formed of portions of at least two different promoters.

According to an embodiment of the present invention, the heterologous polynucleotide is expressed under the transcriptional control of a developmentally regulated promoter specifically active in the plant cell wall upon secondary cell wall deposit.

Thus, the GE and/or AXE expression constructs of the present invention are typically constructed using a developmentally regulated promoter which is specifically active in the plant cell wall upon secondary cell wall deposit.

As used herein, the phrase “developmentally regulated promoter” refers to a promoter capable of directing gene expression at a specific stage of plant growth or development.

As used herein, the phrase “secondary cell wall deposit” refers to the stage during secondary cell wall formation in which developing xylem vessels deposit cellulose at specific sites at the plant plasma membrane.

Thus, the plant promoter employed can be a constitutive promoter, a tissue specific promoter, an inducible promoter, a chimeric promoter or a developmentally regulated promoter.

Examples of constitutive plant promoters include, without being limited to, CaMV35S and CaMV19S promoters, FMV34S promoter, sugarcane bacilliform badnavirus promoter, CsVMV promoter, Arabidopsis ACT2/ACT8 actin promoter, Arabidopsis ubiquitin UBQ1 promoter, barley leaf thionin BTH6 promoter, and rice actin promoter.

The inducible promoter is a promoter induced by a specific stimuli such as stress conditions comprising, for example, light, temperature, chemicals, drought, high salinity, osmotic shock, oxidant conditions or in case of pathogenicity and include, without being limited to, the light-inducible promoter derived from the pea rbcS gene, the promoter from the alfalfa rbcS gene, the promoters DRE, MYC and MYB active in drought; the promoters INT, INPS, prxEa, Ha hsp17.7G4 and RD21 active in high salinity and osmotic stress, and the promoters hsr203J and str246C active in pathogenic stress.

The promoter utilized by the present invention may comprise a strong constitutive promoter such that over expression of the construct inserts is effected following plant transformation.

As mentioned, the promoter utilized by the present invention is preferably a developmentally regulated promoter such that expression is effected in the plant cell wall upon secondary cell wall deposit. Such promoters include, but are not limited to, 4c1 (e.g. 4c1-1), CesA1 (e.g. Eucalyptus grandis cellulose synthase CesA1, e.g. SEQ ID NO: 31), CesA7 (e.g. Eucalyptus grandis CesA7, e.g. SEQ ID NO: 32), CesA8, IRX3 (e.g. SEQ ID NO: 30), IRX4, IRX10 (e.g. SEQ ID NO: 29), DOT1, and FRA8 (e.g. SEQ ID NO: 21) promoters.

According to a specific embodiment, the promoter utilized by the present invention is a FRA8 promoter. An exemplary FRA8 promoter is as set forth in SEQ ID NO: 21.

According to a specific embodiment, the nucleic acid construct comprises a polynucleotide encoding a heterologous acetylxylan esterase (AXE) enzyme as set forth in SEQ ID NO: 1 under the transcriptional control of a FRA8 promoter.

According to a specific embodiment, the nucleic acid construct comprises a polynucleotide encoding a heterologous acetylxylan esterase (AXE) enzyme as set forth in SEQ ID NO: 13 under the transcriptional control of a FRA8 promoter.

According to a specific embodiment, the nucleic acid construct comprises a polynucleotide encoding a heterologous glucuronoyl esterase (GE) enzyme as set forth in SEQ ID NO: 7 under the transcriptional control of a FRA8 promoter.

According to a specific embodiment of the present invention, the AXE and/or GE polynucleotides are expressed in a tissue specific manner.

Thus, the AXE and/or GE enzyme polypeptide expression is targeted to specific tissues of the transgenic plant such that these cell wall-modifying enzymes are present in only some plant tissues during the life of the plant. For example, tissue specific expression may be performed to preferentially express AXE and/or GE enzymes in leaves and stems rather than grain or seed. Tissue-specific expression has other benefits including targeted expression of enzyme(s) to the appropriate substrate.

Tissue specific expression may be functionally accomplished by introducing a constitutively expressed gene in combination with an antisense gene that is expressed only in those tissues where the gene product (e.g., AXE and/or GE enzyme polypeptide) is not desired. For example, a gene coding for AXE and/or GE enzyme polypeptide may be introduced such that it is expressed in all tissues using the 35S promoter from Cauliflower Mosaic Virus. Expression of an antisense transcript of the gene in maize kernel, using for example a zein promoter, would prevent accumulation of the AXE and/or GE enzyme polypeptide in seed. Hence the enzyme encoded by the introduced gene would be present in all tissues except the kernel.

Moreover, several tissue-specific regulated genes and/or promoters may be used according to the present teachings such as those which have been previously reported in plants. Some reported tissue-specific genes include the genes encoding the seed storage proteins (such as napin, cruciferin, .beta.-conglycinin, and phaseolin) zein or oil body proteins (such as oleosin), or genes involved in fatty acid biosynthesis (including acyl carrier protein, stearoyl-ACP desaturase, and fatty acid desaturases (fad 2-1)), and other genes expressed during embryo development, such as Bce4 (Kridl et al., Seed Science Research, 1991, 1: 209). Examples of tissue-specific promoters, which have been described, include the lectin (Vodkin, Prog. Clin. Biol. Res., 1983, 138: 87; Lindstrom et al., Der. Genet., 1990, 11: 160), corn alcohol dehydrogenase 1 (Dennis et al., Nucleic Acids Res., 1984, 12: 983), corn light harvesting complex (Bansal et al., Proc. Natl. Acad. Sci. USA, 1992, 89: 3654), corn heat shock protein, pea small subunit RuBP carboxylase, Ti plasmid mannopine synthase, Ti plasmid nopaline synthase, petunia chalcone isomerase (van Tunen et al., EMBO J., 1988, 7:125), bean glycine rich protein 1 (Keller et al., Genes Dev., 1989, 3: 1639), truncated CaMV 35S (Odell et al., Nature, 1985, 313: 810), potato patatin (Wenzler et al., Plant Mol. Biol., 1989, 13: 347), root cell (Yamamoto et al., Nucleic Acids Res., 1990, 18: 7449), maize zein (Reina et al., Nucleic Acids Res., 1990, 18: 6425; Kriz et al., Mol. Gen. Genet., 1987, 207: 90; Wandelt et al., Nucleic Acids Res., 1989, 17 2354), PEPCase, R gene complex-associated promoters (Chandler et al., Plant Cell, 1989, 1: 1175), chalcone synthase promoters (Franken et al., EMBO J., 1991, 10: 2605), pea vicilin promoter (Czako et al., Mol. Gen. Genet., 1992, 235: 33), bean phaseolin storage protein promoter, DLEC promoter, PHS promoter, zein storage protein promoter, conglutin gamma promoter from soybean, AT2S1 gene promoter, ACT11 actin promoter from Arabidopsis and napA promoter from Brassica napus.

According to an embodiment of the present invention, the tissue comprises the above ground portions of trees and plants including, but not limited to, stems including branches, trunks etc., leaves, blades or any other biomass feedstock components.

According to another embodiment of the present invention, the tissue comprises a xylem or a phloem.

The nucleic acid construct of the present invention may also comprise an additional nucleic acid sequence encoding a signal peptide fused in frame to the heterologous polynucleotide encoding the aforementioned enzyme(s) to allow transport of the AXE or GE propeptides to the endoplasmic reticulum (ER) and through the secretory pathway to the cell wall. Such a signal peptide is typically linked in frame to the amino terminus of a polypeptide (i.e. upstream thereto) and directs the encoded polypeptide into a cell's secretory pathway and its final secretion therefrom (e.g. to the plant cell wall).

Exemplary secretion signal sequences which may be used in accordance with the present teachings, include but are not limited to, the Arabidopsis endoglucanase cell signal peptide (e.g. SEQ ID NO: 22), the Arabidopsis thaliana Expansin-like A1 (e.g. SEQ ID NO: 23), the Arabidopsis thaliana Xyloglucan endotransglucosylase/hydrolase protein 22 (e.g. SEQ ID NO: 24), the Arabidopsis thaliana Pectinesterase/pectinesterase inhibitor 18 (e.g. SEQ ID NO: 25), the Arabidopsis thaliana extensin-like protein 1 (e.g. SEQ ID NO: 26), the Arabidopsis thaliana Laccase-15 (e.g. SEQ ID NO: 27) and the Populus alba Endo-1,4-beta glucanase (e.g. SEQ ID NO: 28).

According to an embodiment, the signal sequence comprises the Arabidopsis endoglucanase cell signal peptide (e.g. SEQ ID NO: 22).

Additional exemplary signal peptides that may be used herein include the tobacco pathogenesis related protein (PR-S) signal sequence (Sijmons et al., 1990, Bio/technology, 8:217-221), lectin signal sequence (Boehn et al., 2000, Transgenic Res, 9(6):477-86), signal sequence from the hydroxyproline-rich glycoprotein from Phaseolus vulgaris (Yan et al., 1997, Plant Phyiol. 115(3):915-24 and Corbin et al., 1987, Mol Cell Biol 7(12):4337-44), potato patatin signal sequence (Iturriaga, G et al., 1989, Plant Cell 1:381-390 and Bevan et al., 1986, Nuc. Acids Res. 41:4625-4638.) and the barley alpha amylase signal sequence (Rasmussen and Johansson, 1992, Plant Mol. Biol. 18(2):423-7).

It will be appreciated that any of the construct types used in the present invention can be co-transformed into the same plant using same or different selection markers in each construct type. Alternatively the first construct type can be introduced into a first plant while the second construct type can be introduced into a second isogenic plant, following which the transgenic plants resultant therefrom can be crossed and the progeny selected for double transformants. Further self-crosses of such progeny can be employed to generate lines homozygous for both constructs.

As mentioned, the expression constructs of the present invention may be generated to comprise only AXE polynucleotides, only GE polynucleotides or to comprise both AXE and GE enzymes.

An exemplary polynucleotide encoding the AXE enzyme of the present invention is as set forth in SEQ ID NOs: 1, 3, 5 or 13.

An exemplary polynucleotide encoding the GE enzyme of the present invention is as set forth in SEQ ID NOs: 7, 9 or 11.

Nucleic acid sequences of the polypeptides of the present invention may be optimized for plant expression. Examples of such sequence modifications include, but are not limited to, an altered G/C content to more closely approach that typically found in the plant species of interest, and the removal of codons atypically found in the plant species commonly referred to as codon optimization.

The phrase “codon optimization” refers to the selection of appropriate DNA nucleotides for use within a structural gene or fragment thereof that approaches codon usage within the plant of interest. Therefore, an optimized gene or nucleic acid sequence refers to a gene in which the nucleotide sequence of a native or naturally occurring gene has been modified in order to utilize statistically-preferred or statistically-favored codons within the plant. The nucleotide sequence typically is examined at the DNA level and the coding region optimized for expression in the plant species determined using any suitable procedure, for example as described in Sardana et al. (1996, Plant Cell Reports 15:677-681). In this method, the standard deviation of codon usage, a measure of codon usage bias, may be calculated by first finding the squared proportional deviation of usage of each codon of the native gene relative to that of highly expressed plant genes, followed by a calculation of the average squared deviation. The formula used is: 1 SDCU=n=1 N [(Xn−Yn)/Yn]2/N, where Xn refers to the frequency of usage of codon n in highly expressed plant genes, where Yn to the frequency of usage of codon n in the gene of interest and N refers to the total number of codons in the gene of interest. A table of codon usage from highly expressed genes of dicotyledonous plants is compiled using the data of Murray et al. (1989, Nuc Acids Res. 17:477-498).

One method of optimizing the nucleic acid sequence in accordance with the preferred codon usage for a particular plant cell type is based on the direct use, without performing any extra statistical calculations, of codon optimization tables such as those provided on-line at the Codon Usage Database through the NIAS (National Institute of Agrobiological Sciences) DNA bank in Japan (www.kazusa.or.jp/codon/). The Codon Usage Database contains codon usage tables for a number of different species, with each codon usage table having been statistically determined based on the data present in Genbank.

By using the above tables to determine the most preferred or most favored codons for each amino acid in a particular species (for example, rice), a naturally-occurring nucleotide sequence encoding a protein of interest can be codon optimized for that particular plant species. This is effected by replacing codons that may have a low statistical incidence in the particular species genome with corresponding codons, in regard to an amino acid, that are statistically more favored. However, one or more less-favored codons may be selected to delete existing restriction sites, to create new ones at potentially useful junctions (5′ and 3′ ends to add signal peptide or termination cassettes, internal sites that might be used to cut and splice segments together to produce a correct full-length sequence), or to eliminate nucleotide sequences that may negatively effect mRNA stability or expression.

The naturally-occurring encoding nucleotide sequence may already, in advance of any modification, contain a number of codons that correspond to a statistically-favored codon in a particular plant species. Therefore, codon optimization of the native nucleotide sequence may comprise determining which codons, within the native nucleotide sequence, are not statistically-favored with regards to a particular plant, and modifying these codons in accordance with a codon usage table of the particular plant to produce a codon optimized derivative. A modified nucleotide sequence may be fully or partially optimized for plant codon usage provided that the protein encoded by the modified nucleotide sequence is produced at a level higher than the protein encoded by the corresponding naturally occurring or native gene. Construction of synthetic genes by altering the codon usage is described in for example PCT Patent Application 93/07278.

Thus, the present invention encompasses nucleic acid sequences described hereinabove; fragments thereof, sequences hybridizable therewith, sequences homologous thereto, sequences orthologous thereto, sequences encoding similar polypeptides with different codon usage, altered sequences characterized by mutations, such as deletion, insertion or substitution of one or more nucleotides, either naturally occurring or man induced, either randomly or in a targeted fashion.

Plant cells may be transformed stably or transiently with the nucleic acid constructs of the present invention. In stable transformation, the nucleic acid molecule of the present invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S, and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledenous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by the present invention.

Transient transformation can be effected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of the present invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that said sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsulated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of the present invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

It will be appreciated that AXE and GE enzymes of the present invention may be co-expressed in a single plant or alternatively may be expressed in two separate plants. If the enzymes are expressed in two separate plants, these plants may be bred in order to obtain a plant co-expressing the two enzymes.

Thus, according to an embodiment of the present invention, a first plant expressing AXE can be crossed with a second plant expressing GE.

It should be noted that although the above described plant breeding approaches utilize two individually transformed plants, approaches which utilize three or more individually transformed plants, each expressing one or two components can also be utilized.

One of ordinary skill in the art would be well aware of various plant breeding techniques and as s such no further description of such techniques is provided herein.

Although plant breeding approaches may be used, it should be noted that a single plant expressing both AXE and GE can be generated via several transformation events each designed for introducing one more expressible components into the cell. In such cases, stability of each transformation event can be verified using specific selection markers.

In any case, transformation and plant breeding approaches can be used to generate any plant and expressing any number of components.

Progeny resulting from breeding or alternatively multiple-transformed plants can be selected, by verifying presence of exogenous mRNA and/or polypeptides by using nucleic acid or protein probes (e.g. antibodies). Alternatively, expression of the enzymes of the present invention may be verified by measuring cell wall acetylation, by measuring the amount of ester linkages between lignin and hemicelluloses or by measuring saccharification yield and pulping efficiency of the transformed plants compared to non-transformed plants of the same type (as described in detail in Example 1 of the Examples section which follows).

According to the present teachings, progeny resulting from breeding or transformation may also be selected by plant physiological characterization monitoring e.g. the growth rate, posture, total weight, dry weight and/or the flowering time of the transgenic plants compared to untransformed plants of the same species.

Once AXE, GE or co-expressing progeny are identified, such plants are further cultivated under conditions which maximize expression of the modifying enzymes and/or the biomass of the crop.

Thus, AXE, GE or co-expressing progeny can be grown under different conditions suitable for optimal biomass production of each species.

A person of ordinary skill in the art is capable of determining if to generate a plant expressing a single enzyme (i.e. AXE or GE) or a plant co-expressing both AXE and GE, especially in light of the detailed disclosure provided herein. It will be appreciated that the type of plant and its intended use need to be taken into account when making such a decision, as in some plants expression of a single enzyme will enable high saccharification and digestibility, wherein in other plants co-expression may be needed in order to improve saccharification and digestibility of the plant.

The present invention provides methods of producing a plant having reduced acetylation and reduced lignin hemicellulose ester crosslinks in a cell wall of the plant. The method comprising: (a) expressing in a first plant a heterologous polynucleotide encoding an acetylxylan esterase (AXE) enzyme under the transcriptional control of a developmentally regulated promoter specifically active in the cell wall upon secondary cell wall deposit; (b) expressing in a second plant a heterologous polynucleotide encoding a glucuronoyl esterase (GE) enzyme under the transcriptional control of a developmentally regulated promoter specifically active in the cell wall upon secondary cell wall deposit; and (c) crossing the first plant and the second plant and selecting progeny expressing the acetylxylan esterase (AXE) enzyme and the glucuronoyl esterase (GE) enzyme, thereby producing the plant having the reduced acetylation and the reduced lignin hemicellulose ester crosslinks in the cell wall.

The present invention further provides methods of producing a plant having reduced acetylation and reduced lignin hemicellulose ester crosslinks in a cell wall of the plant. The method comprising: (a) expressing in a first plant a heterologous polynucleotide encoding an acetylxylan esterase (AXE) enzyme as set forth in SEQ ID NOs: 2, 4, 6 or 14; (b) expressing in a second plant a heterologous polynucleotide encoding a glucuronoyl esterase (GE) enzyme as set forth in SEQ ID NOs: 8, 10 or 12; and (c) crossing the first plant and the second plant and selecting progeny expressing the acetylxylan esterase (AXE) enzyme and the glucuronoyl esterase (GE) enzyme, thereby producing the plant having the reduced acetylation and the reduced lignin hemicellulose ester crosslinks in the cell wall.

According to an embodiment of the present invention, there is provided a transformed plant comprising reduced covalent links between a hemicellulose and a lignin in a cell of the plant. Such a plant is generated by expression of a GE enzyme in the cell walls of the plant.

According to another embodiment of the present invention, there is provided a transformed plant comprising reduced acetylation in a cell of the plant. Such a plant is generated by expression of an AXE enzyme in the cell walls of the plant.

According to another embodiment of the present invention, there is provided a transformed plant comprising reduced covalent links between a hemicellulose and a lignin in a cell wall of the plant and comprising reduced acetylation in the cell of the plant. Such a plant is generated by co-expression of a GE enzyme and an AXE enzyme in the cell walls of the plant.

Plants of the present invention with improved saccharification and digestibility of the plant tissues are extensively useful in biomass conversion (e.g. biofuels, hydrogen production), for feed and food applications, and for pulp and paper industries.

As used herein the phrase “plant biomass” refers to biomass that includes a plurality of components found in plants, such as lignin, cellulose, hemicellulose, beta-glucans, homogalacturonans, and rhamnogalacturonans. Plant biomass may be obtained, for example, from a transgenic plant expressing AXE and/or GE essentially as described herein. Plant biomass may be obtained from any part of a plant, including, but not limited to, leaves, stems, seeds, and combinations thereof.

According to an embodiment of the present invention, there is provided a method of producing a biofuel, the method comprising growing the genetically modified AXE and/or GE expressing plant under conditions which allow degradation of lignocellulose to form a hydrolysate mixture, and incubating the hydrolysate mixture under conditions that promote conversion of fermentable sugars of the hydrolysate mixture to ethanol, butanol acetic acid or ethyl acetate.

It will be appreciated that using the genetically modified AXE and/or GE expressing plant of the present invention for production of biofuel requires less pretreatment chemicals than required by a non-transgenic plant of the same species.

It will be appreciated that using the genetically modified AXE and/or GE expressing plant of the present invention for production of biofuel will render the plant biomass more amenable to microbial and/or physical degradation during for example pretreatment processes including storage in silage containers in which the biomass is exposed to microorganisms and/or long term pretreatment regimes including heat and enzymes added to the process resulting in the requirement of less pretreatment chemicals than required by a non-transgenic plant of the same species.

Thus, plants transformed according to the present invention provide a means of increasing biofuel (e.g. ethanol) yields, reducing pretreatment costs by reducing acid/heat pretreatment requirements for saccharification of biomass; and/or reducing other plant production and processing costs, such as by allowing multi-applications and isolation of commercially valuable by-products.

According to another embodiment of the present invention, the AXE and/or GE expressing plant of the present invention may be used for the paper and pulp industries.

In a further aspect the invention, the AXE expressing and/or GE expressing transgenic plants or parts thereof are comprised in a food or feed product (e.g., dry, liquid, paste). A food or feed product is any ingestible preparation containing the AXE expressing and/or GE expressing transgenic plants, or parts thereof, of the present invention, or preparations made from these plants. Thus, the plants or preparations are suitable for human (or animal) consumption, i.e. the AXE expressing and/or GE expressing transgenic plants or parts thereof are more readily digested. Feed products of the present invention further include a beverage adapted for animal consumption.

It will be appreciated that the AXE expressing and/or GE expressing transgenic plants, or parts thereof, of the present invention may be used directly as feed products or alternatively may be incorporated or mixed with feed products for consumption. Exemplary feed products comprising the AXE expressing and/or GE expressing transgenic plants, or parts thereof, include, but are not limited to, grains, cereals, such as oats, e.g. black oats, barley, wheat, rye, sorghum, corn, vegetables, leguminous plants, especially soybeans, root vegetables and cabbage, or green forage, such as grass or hay.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### GENERAL MATERIALS AND EXPERIMENTAL PROCEDURES

Cloning and Transformation of Acetylxylan Esterase (AXE) and Glucuronoyl Esterase (GE), Together or Separately, into Tobacco, Poplar and Eucalyptus Plants

Promoters:

AXE and GE activity during secondary cell wall deposit is achieved by fusing AXE and GE genes with various promoters, including:

(1) Secondary cell wall specific promoters: 4c1 promoter and CesA7 promoter.

(2) Constitutive promoter: 35s promoter; and/or Rubisco promoter.

(3) Xylem specific promoter: IRX4 promoter, FRA8 promoter.

Signal Peptide:

In order to direct the AXE and GE to the cell wall, the respective genes are fused to nucleic acid sequences which encode for a secretion leader peptide, this allows the translated genes to be processed in the ER pathway and to be secreted to the extracellular matrix. An example of a cell wall secretion leader peptide which may be used includes the Arabidopsis endoglucanase cell signal peptide.

Assaying the Activity of the Acetylxylan Esterase (AXE) in Transgenic Compared to Wild Type Plants

AXE activity in plant tissues is assayed by taking 0.5 gram of plant tissue, adding 1 ml sodium phosphate buffer (pH 7.0; 100 mM) and homogenizing with mortar and pestle. Acetylxylan esterase activity is then determined on the prepared extract by measuring the amount of 4-methylumbelliferone released from 4-methylumbelliferyl acetate as follows: sodium phosphate buffer 100 μl (pH 7.0; 100 mM) and 240 μl H₂O are preincubated at 50° C. for 12 min. 50 μl plant extract is added to the buffer, and the reaction is initiated within 1 min by adding 10 μl of 100 mM 4-methylumbelliferyl acetate in dimethyl sulfoxide. After 2 to 10 min, the reaction is stopped by adding 600 μl of 50 mM citric acid. Absorbance is determined at 354 nm.

Measuring Cell Wall Acetylation

Cell walls are isolated from plant material by the following method:

1. Take 100 mg dry stem ground to fine powder.

2. Add 1 ml of 70% ethanol. Vortex and shortly centrifuge (i.e. spindown) and discard the resultant supernatant.

3. Add 1 ml chloroform:methanol (at a 1:1 ratio). Vortex and shortly centrifuge (i.e. spindown) and discard the resultant supernatant.

4. Dry the sample by adding 500 μl acetone and air drying.

5. De-starch by incubating the dry pellet with 35 μl α-amylase (50 μg/1 mL; from Bacillus species); 17 μl Pullulanase (18.7 units from bacillus acidopullulyticus). Cap the tube and vortex thoroughly.

6. Incubate overnight at 37° C.

7. Heat the suspension at 100° C. for 10 min in a heating block to terminate digestion. Centrifuge (10,000 rpm, 10 min) and discard the supernatant which contains the solubilized starch.

8. Wash with water and then 3 times with acetone. Dry the pellet using an air drier. This pellet is the cell wall material (CWM).

Duplicates are weighed and 10 mg samples CWM are placed into a centrifuge tube fitted with gas-tight cap or lid.

1 ml isopropanol/NaOH solution (4° C.) is added to each tube. Tubes are capped and mixed gently. Mixture is left to stand for 2 hrs at room temperature and then centrifuged for 10 min at 2,000×g (at room temperature). Supernatant is removed and placed in a small vial with a septum. Vial is immediately sealed.

15 μl of sample is injected into an HPLC system equipped with rezex RHM-Monosaccharide column and a 5 mM H₂SO₄ solvent system is used, set at a flow rate of 0.6 ml/min and a temperature of 30° C. The refractive index detector is set at 40° C.

Assaying the Activity of the Glucuronoyl Esterase (GE) in Transgenic Compared to Wild Type Plants

GE activity in plant tissues is assayed by taking 0.5 gram of plant tissue, adding 1 ml sodium phosphate buffer (pH 6.0, 50 mM) and homogenizing with mortar and pestle. Quantitative glucuronoyl esterase assay is based on the measurement of the decrease in 4-nitrophenyl 2-O-(methyl 4-O-methyl-α-D-glucopyranosyluronate)-β-D-xylopyranoside concentration due to de-esterification. The ester (2 mM) is incubated with the plant extract in sodium phosphate buffer (pH 6.0, 50 mM) at 30° C. and its concentration is monitored over time by HPLC on a C18, 7 μm column (250×4 mm) eluted with acetonitrile:water (2:1, v/v) using a UV-detector operating at 308 nm. One unit of glucuronoyl esterase activity is defined as the amount of the enzyme deesterifying 1 μmol of 4-nitrophenyl 2-O-(methyl 4-O-methyl-α-D-glucopyranosyluronate)-β-D-xylopyranoside in 1 min at 30° C.

Measuring the Amount of Ester Linkages Between Lignin and Hemicelluloses

FT-IR spectra of biomass samples are obtained on an FT-IR spectrophotometer using a KBr disk containing 1% finely ground samples. Thirty-two scans are taken of each sample recorded from 4000 to 400 cm⁻¹ at a resolution of 2 cm⁻¹ in the transmission mode. A change in the peak at ˜1730 cm⁻¹ is correlated with the amount of uronic and ester groups or the ester binds of the carboxylic groups of ferulic and/or p-coumaric acids.

Selection of the Best Performing DNA Constructs in Terms of Saccharification, Pulping Efficiency and Normal Growth

Saccharification (sugar release) assay protocol:

1) To 200 mg dry biomass add 1.8 ml 1% H₂SO₄.

2) Autoclave at 120° C. for 20 min (to get a brown syrup).

3) Wash and filter (glass filter paper) with 30 ml double distilled water.

4) Add 1 ml cellulase (10 FPU\ml in 50 mM citrate buffer, pH 4.8)+1.5 ml citrate buffer (1 M, pH 4.8)+15 μl thymol (50 g\l in 70% ethanol)+12.5 ml double distilled water.

5) Incubate at 45° C. at 125 rpm.

6) Add 100 μl glucose standards or the saccharification sup to 1000 μl of Dinitrosalisylic acid. Incubate at 100° C. for 10 min. Measure the absorbance at 540 nm.

Molecular, Biochemical and Physiological Characterization of Transgenic Tobacco Plants

Characterization of Cell Wall Structure and Composition:

A. Determination and analysis of sugar content and composition by ion exchange

HPLC Rezex Pb²⁺ RPM Column:

1) Cell wall is isolated by taking 100 mg dry stem ground to fine powder.

2) Add 1 ml of 70% ethanol. Vortex, shortly centrifuge (i.e. spindown) and discard the resultant supernatant.

3) Add 1 ml chlorophorm:methanol (at a 1:1 ratio). Vortex, shortly centrifuge (i.e. spindown) and discard the resultant supernatant.

4) Dry the sample by adding 500 μl acetone and air dry.

5) De-starch by incubating the dry pellet with 35 μl α-amylase (50 μg/1 mL; from Bacillus species); 17 μl Pullulanase (18.7 units from bacillus acidopullulyticus). Cap the tube and vortex thoroughly.

6) Incubate overnight at 37° C.

7) Heat the suspension at 100° C. for 10 min in a heating block to terminate digestion. Centrifuge (10,000 rpm, 10 min) and discard the supernatant which contains the solubilized starch.

8) Wash with water and then 3 times with acetone. Dry the pellet using an air drier. This pellet is the cell wall material (CWM)

9) Take 10 mg of CWM and add 125 ul of 72% (w/w) sulfuric acid at room temp, incubate for one hour.

10) Add 1.35 ml of double distilled water and incubate at 100° C. for 2 hours.

11) Add 150 mg of calcium carbonate to neutralize the solution.

12) Apply 10-50 μl of the hydrolyzed cell walls to HPLC system equipped with Rezex Pb²⁺ RPM column and refractive index. Flow rate-0.6 ml/minute. Use the following standards: cellobiose, glucose, xylose, arabinose, galactose and mannose.

B. Cell wall structure is analyzed by hand cutting the stem sections and analyzing using RAMAN microscopy analysis.

C. Cell wall ultrastructure and morphology is analyzed by screening sections in Scanning Electron Microscopy (SEM) and Transmission Electron microscopy (TEM).

Plant Physiological Characterization

Physiological characterization is performed in a greenhouse facility monitoring growth rate, total weight, dry weight and flowering time of the transgenic plants compared to wild type plants.

### Example 1

### Cloning and Transformation of Acetylxylan Esterase (AXE) and Glucuronoyl Esterase (GE) into Tobacco Plants

Promoters:

Since both AXE and GE over-expression within the plant may reduce plant structural integrity and fitness, inventors directed the expression of these enzymes to specific developmental stages such as secondary cell wall development or xylem cells development. Expression of a gene at a specific developmental stage can be done by developmentally specific promoters. Examples for such promoters, for example promoters that are expressed only during secondary wall-thickening, are CesA7 promoter and 4CL-1 promoter. Examples for promoters that are expressed in xylem tissue development are FRA8 promoter and DOT1 promoter.

To achieve expression at the xylem developmental stage AXE and GE were fused to the FRA8 promoter (SEQ ID NO: 21).

Constitutive over-expression of AXE by CaMV 35S promoter was also tested.

Signal Peptide:

In order to direct the AXE and GE to the cell wall, the respective genes were fused to a cell wall specific leader peptide, this allows the genes to be translated in the ER pathway and to be secreted to the extracellular matrix. An example of a cell wall specific signal peptide which may be used includes the Arabidopsis endoglucanase cell signal peptide (SEQ ID NO: 22).

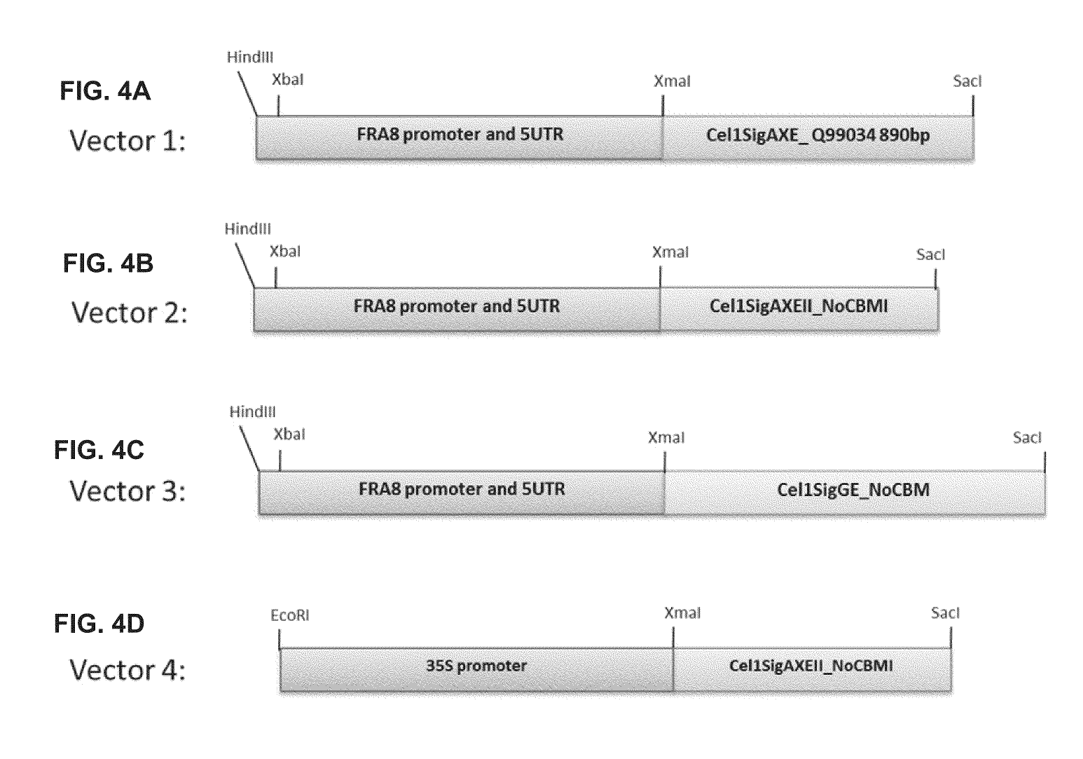

As illustrated in FIGS. 4A-D, four transformation vectors were constructed, namely:

Vector no. 1—FRA8 promoter::AXEI (SEQ ID NO: 1).

Vector no. 2—FRA8 promoter::AXEII (SEQ ID NO: 13).

Vector no. 3—FRA8 promoter::GE (SEQ ID NO: 7).

Vector no. 4—35S promoter::AXEII (SEQ ID NO: 13).

### Example 2

### Tobacco Transformation and PCR to Genomic DNA

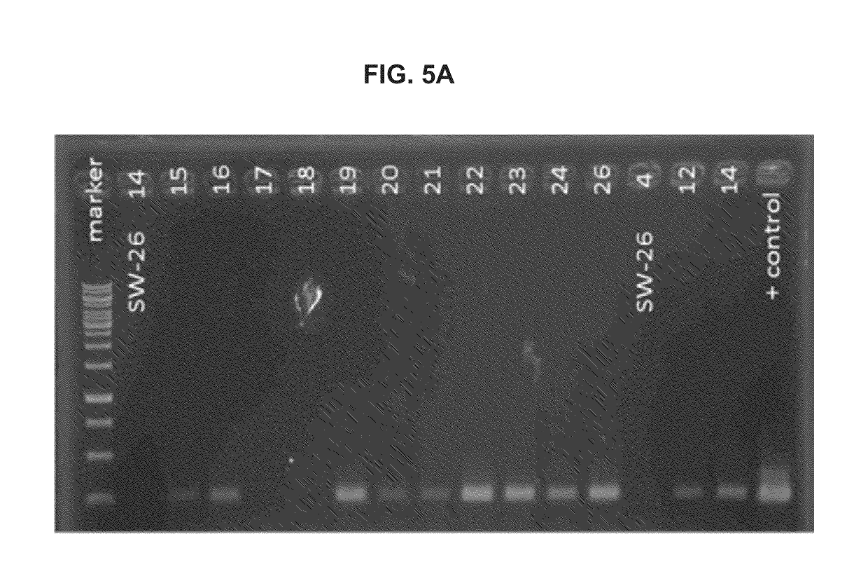

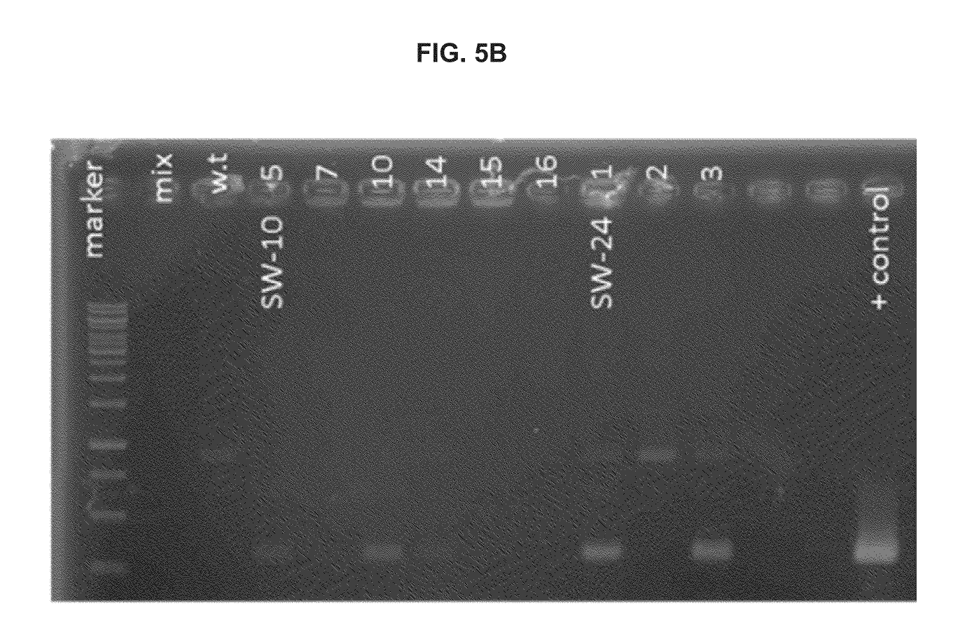

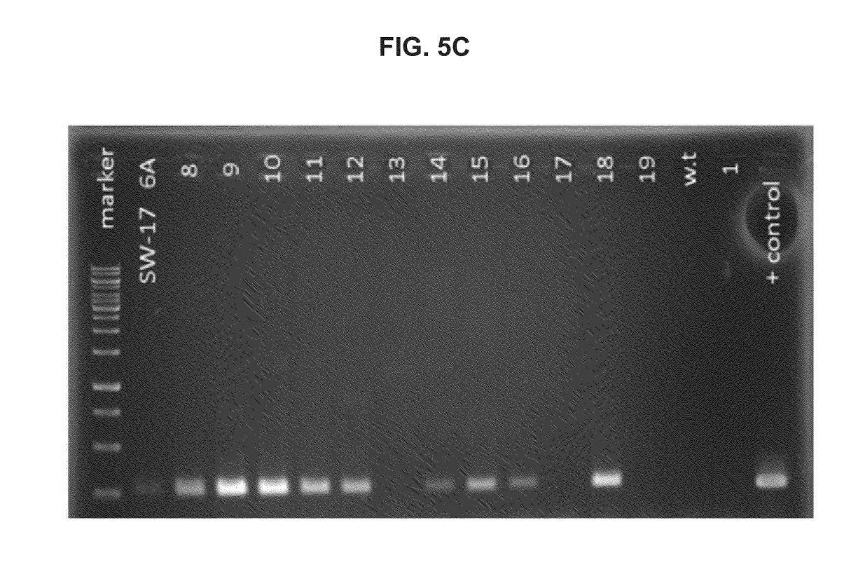

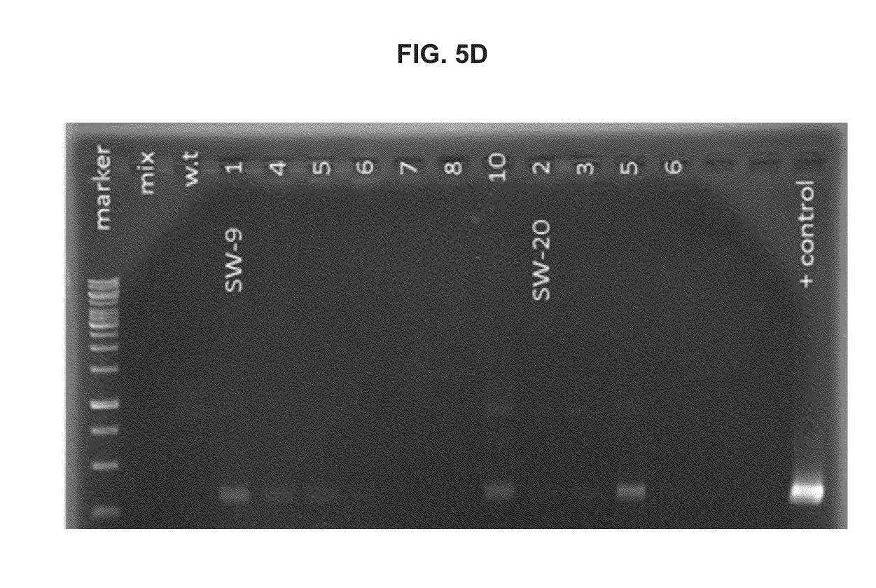

Leaf-disc transformation was performed with Nicotiana tabacum-SR1 plants as described previously [Block, M. D. et al., EMBO Journal (1984) 3: 1681-1689]. More than 15 independent tobacco transformants were generated for each binary vector, propagated in vitro and transferred to the greenhouse. Tobacco plants over-expressing AXEII under the control of the 35S promoter flowered earlier and showed various levels of modified phenotype, such as retarded growth and lower stem caliber (data not shown), as compared to plants expressing AXEII or AXEI under the control of the FRA8 promoter or wild type plants (untransformed plants grown under the same growth conditions). The presence of the transgene was confirmed by western blot analysis to the nptII protein (data not shown) and by PCR (FIGS. 5A-D) on genomic DNA using specific primers for AXE or GE (Table 1). The binary vectors were used as a template for positive control.

TABLE 1
PCR primers:

| | | Prod- |
|---|---|---|
| | | uct |
| Gene | Primers | size |
| AXEI | Forward TGGTGCTAGTCGGGTATTCTCAAG | Ap- |
| (SEQ | (SEQ ID NO: 15) | proxi- |
| ID | Reverse TAGATGCACTAGGACACACGAACC | mate- |
| NO: | (SEQ ID NO: 16) | ly |
| 1) | | 250 bp |
| AXEII | Forward CAATTCCTTCAACTCGCAGTGTCC | Ap- |
| (SEQ | (SEQ ID NO: 17) | proxi- |
| ID | Reverse GCGTCGCAATAGCTCTTGATCTTG | mate- |
| NO: | (SEQ ID NO: 18) | ly |
| 13 | | 300 bp |
| GE | Forward GTTGCTCCAAGACCGTTGATAAGC | Ap- |
| (SEQ | (SEQ ID NO: 19) | proxi- |
| ID | Reverse AGAGTCCGTTTGTCTCGAAGATCG | mate- |
| NO: | (SEQ ID NO: 20) | ly |
| 7) | | 250 bp |

### Example 3

### Transcription Analysis of the Transgenic Plants

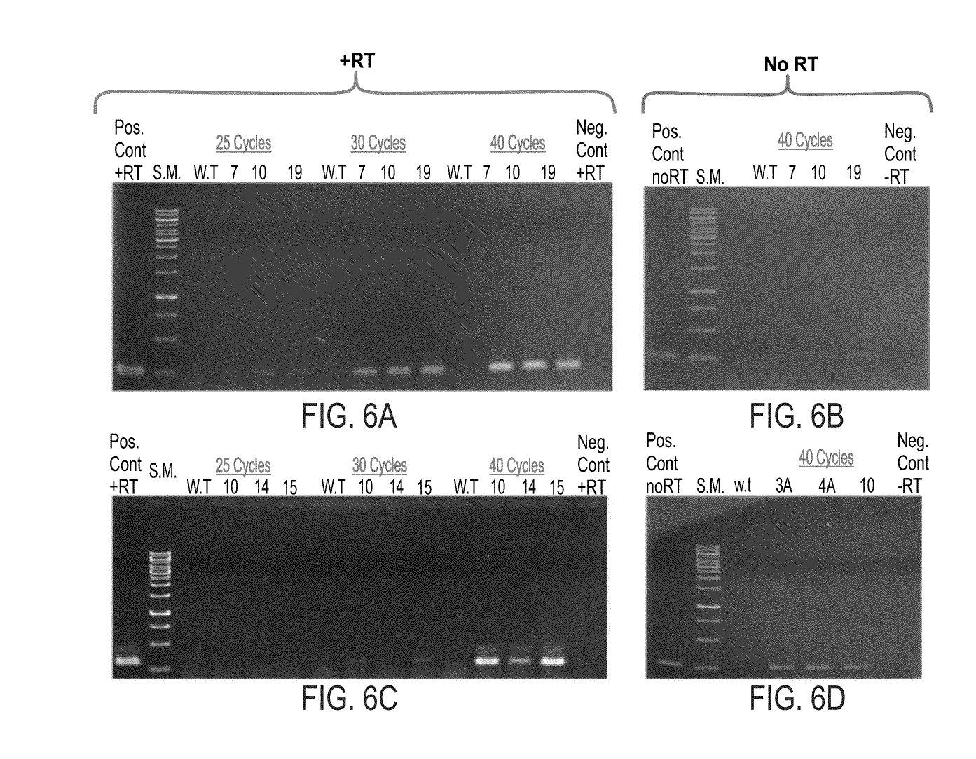

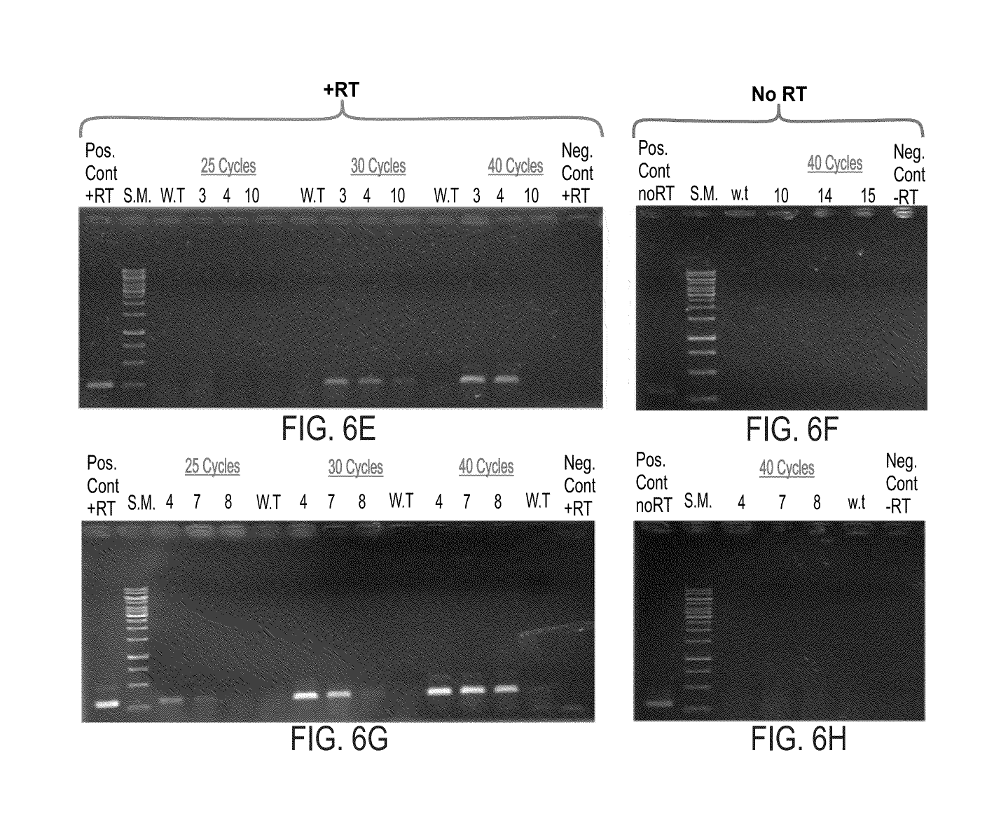

AXE and GE expression patterns were examined by RT-PCR (FIGS. 6A-H). Total RNA was isolated from leaves of tobacco plants. DNA removed by DNAse. PCR was performed using cDNA from the first-strand reaction with primers specific for the AXE and GE (see Table 1, above). The binary vectors were used as a template for positive control. To confirm negative DNA contamination PCRs were generated without reverse transcriptase.

### Example 4

### Activity Assay to AXE Plants

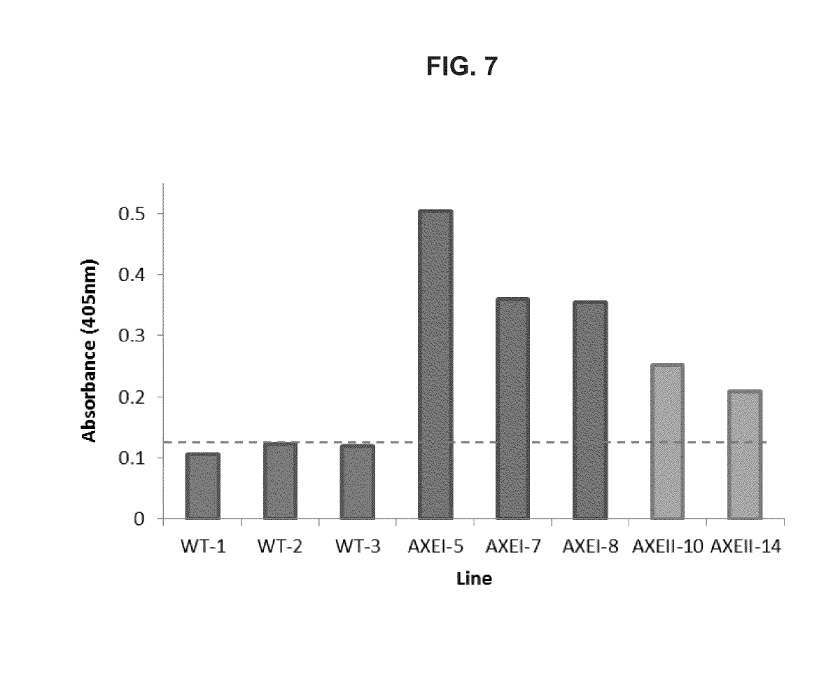

Acetylxylan esterase activity was measured by incubation of crude extract of tobacco leaves in 2000 μl of reaction mixture containing 0.55 mM of pNP-acetyl (Sigma, N8130) in 50 mM sodium citrate buffer pH 5.9. Two negative controls were used: the reaction mixture without plant extract and plant extract only without substrate. The reactions were carried out at ambient temperature and terminated at different time points. Absorbance was measured at 405 nm on a microplate reader. FIG. 7 indicates that both AXEI and AXEII proteins were active in the transgenic plants.

### Example 5

### Quantitative Measurement of Acetyl Groups

For the measurement of acetic acid release, cell wall material (CWM) was prepared and enzymatically destarched according to the method previously described [Foster, C. E., Martin, T. M. & Pauly, M. Comprehensive compositional analysis of plant cell walls (Lignocellulosic biomass) part I: lignin. Journal of visualized experiments: JoVE 5-8 (2010).doi:10.3791/1745]. CWM (10 mg) was saponified in 550 μl of 0.09 M NaOH at ambient temperature overnight. The sample was neutralized by adding 52 μl 1M HCl. The suspension was centrifuged at 12,000×rpm for 10 min immediately before the measurement of acetic acid. Acetic acid was determined using HPLC system equipped with rezex ROA-organic acid column. Filtered aliquots of 10 μL were injected on HPLC operating at a flow rate of 0.6 mL/min and the HPLC column was heated to 65° C.

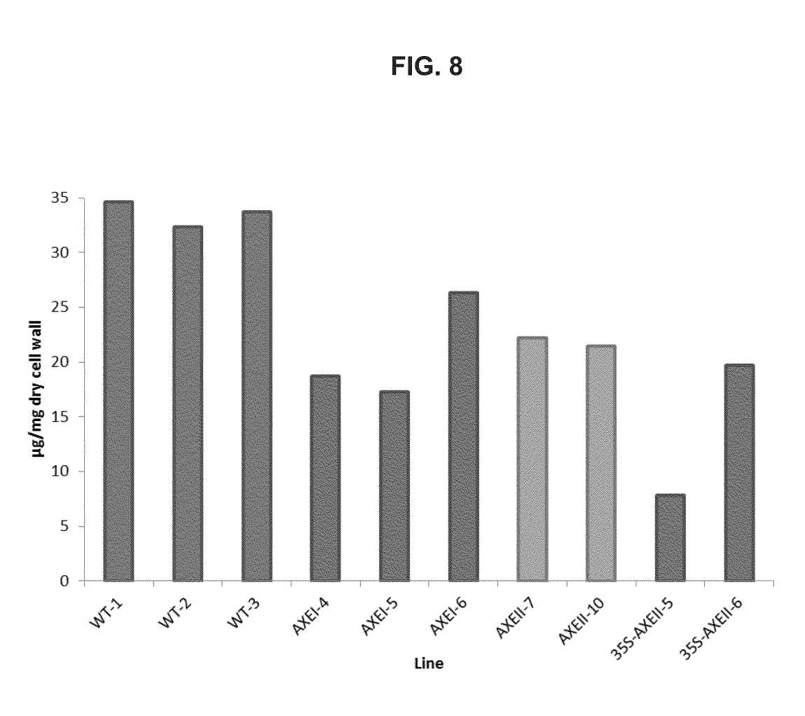

Quantitative analysis of the acetyl groups released from the CWM revealed up to a 75% reduction in the 35S::AXEII plants and up to 50% in the FRA8::AXEI plants compared with the wild type (FIG. 8).

### Example 6

### Saccharification of Transgenic Plants

Four week old stems were dried at 65° C. overnight, ground to fine powder and screened thru 1 mm sieve. 60 mg of dry powder were mixed with 500 μL of water and autoclaved at 120° C. for 15 minutes. Saccharification was initiated by adding 1000 μL of 75 mM sodium citrate buffer pH 5 containing 0.045% w/v sodium azide, 3.75% v/v Celluclast 1.5 L (Sigma C2730) and 0.25% w/w β-glucosidase (Novozymes). After 24 h of incubation at 50° C. with shacking (250 rpm), samples were centrifuged (12,000 rpm, 5 min) diluted×10 and 100 μl of supernatant tested for reducing sugar content using the DNS assay and glucose solutions as standards previously described [Ghose, T. K. Measurment of cellulase activities. Pure & Appl. Chem (1987) 59, 257-268].

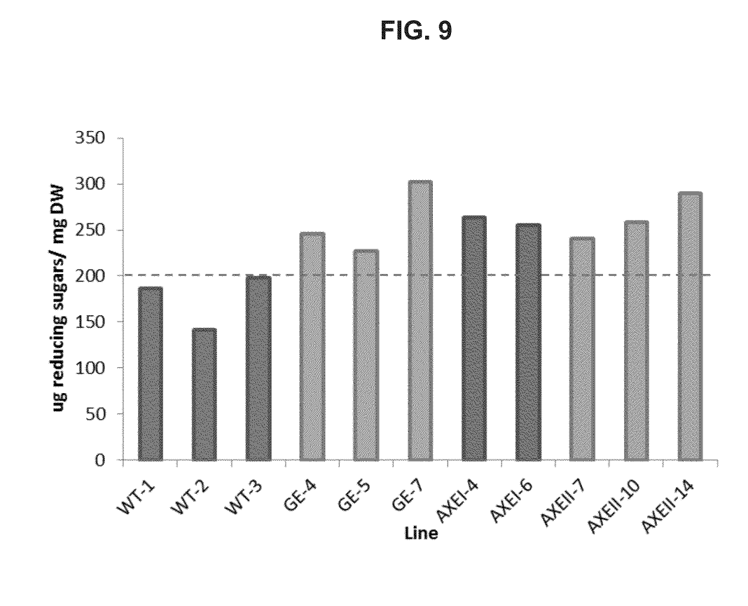

As illustrated in FIG. 9, hot water treated biomass of AXE and GE expressing plants released more reducing sugars compared to wild type. Improvement of saccharification efficiency observed for the different transgenic plant lines ranged from 5% to 40%.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of engineering a plant having reduced acetylation in a cell wall, the method comprising expressing in the plant cell wall at least one isolated heterologous polynucleotide encoding an acetylxylan esterase (AXE) enzyme under the transcriptional control of a developmentally regulated promoter specifically active in the plant cell wall upon secondary cell wall deposit, thereby engineering the plant having reduced acetylation in the cell wall.

2. (canceled)

3. A method of engineering a plant having reduced lignin hemicellulose ester crosslinks in a cell wall, the method comprising expressing in the plant cell wall at least one isolated heterologous polynucleotide encoding a glucuronoyl esterase (GE) enzyme under the transcriptional control of a developmentally regulated promoter specifically active in the plant cell wall upon secondary cell wall deposit, thereby engineering the plant having reduced lignin hemicellulose ester crosslinks in the cell wall.

4. (canceled)

5. The method of claim 1, further comprising expressing in the plant an additional heterologous polynucleotide encoding a glucuronoyl esterase (GE) enzyme.

6. The method of claim 3, further comprising expressing in the plant an additional heterologous polynucleotide encoding an acetylxylan esterase (AXE) enzyme.

7. (canceled)

8. The method of claim 1, wherein said AXE enzyme is selected from the group consisting of SEQ ID NO: 2, SEQ ID NO: 4, SEQ ID NO: 6 and SEQ ID NO: 14.

9. The method of claim 3, wherein said GE enzyme is selected from the group consisting of SEQ ID NO: 8, SEQ ID NO: 10 and SEQ ID NO: 12.

10. The method of claim 2, wherein said isolated heterologous polynucleotide is expressed in a tissue specific manner.

11. The method of claim 10, wherein said tissue is selected from the group consisting of a stem and a leaf.

12. The method of claim 10, wherein said tissue comprises a xylem or a phloem.

13. A genetically modified plant expressing a heterologous polynucleotide encoding an acetylxylan esterase (AXE) enzyme under the transcriptional control of a developmentally regulated promoter specifically active in the plant cell wall upon secondary cell wall deposit.

14. (canceled)

15. A genetically modified plant expressing a heterologous polynucleotide encoding a glucuronoyl esterase (GE) enzyme under the transcriptional control of a developmentally regulated promoter specifically active in the plant cell wall upon secondary cell wall deposit.

16. (canceled)

17. A genetically modified plant co-expressing a heterologous polynucleotide encoding an acetylxylan esterase (AXE) enzyme and a heterologous polynucleotide encoding a glucuronoyl esterase (GE) enzyme under the transcriptional control of a developmentally regulated promoter specifically active in the plant cell wall upon secondary cell wall deposit.

18-19. (canceled)

20. The genetically modified plant of claim 13, wherein said AXE enzyme is as set forth in SEQ ID NOs: 2, 4, 6 or 14.

21. The genetically modified plant of claim 15, wherein said GE enzyme is as set forth in SEQ ID NOs: 8, 10 or 12.

22. A plant system comprising:
(i) a genetically modified plant expressing a heterologous polynucleotide encoding an acetylxylan esterase (AXE) enzyme under the transcriptional control of a developmentally regulated promoter specifically active in the plant cell wall upon secondary cell wall deposit; and
(ii) a genetically modified plant expressing a heterologous polynucleotide encoding a glucuronoyl esterase (GE) enzyme under the transcriptional control of a developmentally regulated promoter specifically active in the plant cell wall upon secondary cell wall deposit.

23. A method of producing a plant having reduced acetylation and reduced lignin hemicellulose ester crosslinks in a cell wall, the method comprising:
(a) expressing in a first plant a heterologous polynucleotide encoding an acetylxylan esterase (AXE) enzyme under the transcriptional control of a developmentally regulated promoter specifically active in the cell wall upon secondary cell wall deposit;
(b) expressing in a second plant a heterologous polynucleotide encoding a glucuronoyl esterase (GE) enzyme under the transcriptional control of a developmentally regulated promoter specifically active in the cell wall upon secondary cell wall deposit; and
(c) crossing said first plant and said second plant and selecting progeny expressing said acetylxylan esterase (AXE) enzyme and said glucuronoyl esterase (GE) enzyme, thereby producing the plant having said reduced acetylation and said reduced lignin hemicellulose ester crosslinks in the cell wall.

24. (canceled)

25. The method of claim 23, wherein said heterologous polynucleotide encoding said AXE enzyme is at least 90% homologous to the nucleic acid sequence as set forth in SEQ ID NOs: 1, 3, 5 or 13 and said heterologous polynucleotide encoding said GE enzyme is at least 90% homologous to the nucleic acid sequence as set forth in SEQ ID NOs: 7, 9 or 11.

26. The method of claim 23, wherein said plant comprises reduced covalent links between a hemicellulose and a lignin in a cell of said plant as compared to a non-transgenic plant of the same species.

27. The method of claim 23, wherein said plant comprises reduced acetylation in a cell of said plant as compared to a non-transgenic plant of the same species.

28. The genetically modified plant of claim 13, wherein said plant is selected from the group consisting of a corn, a switchgrass, a sorghum, a miscanthus, a sugarcane, a poplar, a pine, a wheat, a rice, a soy, a cotton, a barley, a turf grass, a tobacco, a bamboo, a rape, a sugar beet, a sunflower, a willow, a hemp, and an eucalyptus.

29. A food, feed or feedstock comprising the genetically modified plant of claim 13.

30. A method of producing a biofuel, the method comprising:
(a) growing the genetically modified plant of claim 13, under conditions which allow degradation of lignocellulose to form a hydrolysate mixture; and
(b) incubating the hydrolysate mixture under conditions that promote conversion of fermentable sugars of the hydrolysate mixture to ethanol, butanol, acetic acid or ethyl acetate, thereby producing said biofuel.

31. The method of claim 30, wherein said conditions comprise less pretreatment chemicals then required by a non-transgenic plant of the same species.

32. A nucleic acid construct comprising:
(i) a polynucleotide encoding a heterologous acetylxylan esterase (AXE) enzyme under the transcriptional control of a developmentally regulated promoter specifically active in the plant cell wall upon secondary cell wall deposit;
(ii) a polynucleotide encoding a heterologous glucuronoyl esterase (GE) enzyme under the transcriptional control of a developmentally regulated promoter specifically active in the plant cell wall upon secondary cell wall deposit; or
(iii) a polynucleotide encoding a heterologous acetylxylan esterase (AXE) enzyme and a polynucleotide encoding a heterologous glucuronoyl esterase (GE) enzyme both under the transcriptional control of a developmentally regulated promoter specifically active in the plant cell wall upon secondary cell wall deposit.

33-34. (canceled)

35. The nucleic acid construct of claim 32, wherein said polynucleotide encoding said AXE enzyme is at least 90% homologous to the nucleic acid sequence as set forth in SEQ ID NOs: 1, 3, 5 or 13.

36. The nucleic acid construct of claim 32, wherein said polynucleotide encoding said GE enzyme is at least 90% homologous to the nucleic acid sequence as set forth in SEQ ID NOs: 7, 9 or 11.

37-39. (canceled)

40. The nucleic acid construct of claim 32, further comprising a nucleic acid sequence encoding a signal peptide capable of directing AXE or GE expression in a plant cell wall.

41. The method of claim 1, wherein said heterologous polynucleotide encoding said AXE enzyme is conjugated to a nucleic acid sequence encoding a signal peptide capable of directing AXE expression in a plant cell wall.

42. The nucleic acid construct of claim 40, wherein said signal peptide is selected from the group consisting of an Arabidopsis endoglucanase cell signal peptide, an Arabidopsis thaliana Expansin-like A1, an Arabidopsis thaliana Xyloglucan endotransglucosylase/hydrolase protein 22, an Arabidopsis thaliana Pectinesterase/pectinesterase inhibitor 18, an Arabidopsis thaliana extensin-like protein 1, an Arabidopsis thaliana Laccase-15 and a Populus alba Endo-1,4-beta glucanase.

43. (canceled)

44. The method of claim 1, wherein said promoter is selected from the group consisting of 4c1, CesA1, CesA7, CesA8, IRX3, IRX4, IRX10, DOT1 and FRA8.

45. (canceled)

46. The method of claim 3, wherein said heterologous polynucleotide encoding said GE enzyme is conjugated to a nucleic acid sequence encoding a signal peptide capable of directing GE expression in a plant cell wall.

47. The method of claim 3, wherein said isolated heterologous polynucleotide is expressed in a tissue specific manner.

48. The method of claim 47, wherein said tissue is selected from the group consisting of a stem and a leaf.

49. The method of claim 47, wherein said tissue comprises a xylem or a phloem.
